(19) United States
(12) Patent Application Publication
(10) Pub. No.: US 2002/0001831 A1
(43) Pub. Date: Jan. 3, 2002

(54) Low cost manufacture of oligosaccharides

(76) Inventors: Shawn Defrees, North Wales, PA (US); Karl Johnson, Willow Grove, PA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/757,289

(22) Filed: Jan. 8, 2001

Related U.S. Application Data
(63) Continuation of application No. 09/442,111, filed on Nov. 17, 1999.
(60) Provisional application No. 60/109,031, filed on Nov. 18, 1998.
(60) Provisional application No. 60/109,096, filed on Nov. 19, 1998.

(51) Int. Cl. C12P 19/26; C12P 19/04; C08B 37/00

(52) U.S. Cl. 435/101; 435/84; 536/53

(57) ABSTRACT

This invention provides recombinant cells, reaction mixtures, and methods that are useful for the enzymatic synthesis of product saccharides. The recombinant cells contain a heterologous gene that encodes a glycosyltransferase which catalyzes at least one step of the enzymatic synthesis, as well as a system for generating a nucleotide sugar that can serve as a substrate for the glycosyltransferase.

### BACKGROUND OF THE INVENTION

1. Field of the Invention

The present invention relates to the enzymatic synthesis of product saccharides, including oligosaccharides. In particular, it relates to the use of cells to express glycosyltransferases and to synthesize reactants that are used in glycosyltransferase-catalyzed saccharide synthesis. The methods make possible the synthesis of complex product saccharides in a single vessel using readily available, relatively inexpensive starting materials.

2. Background

Oligosaccharides, with their branched structure and the multiple linkages with which monomers can be attached to each other, have a greater potential to carry information in a short sequence than any other biological oligomer. The number of isomer permutations for a trisaccharide composed of three hexoses has recently been calculated as being greater than 38,000 (Laine (1994) Glycobiology 4: 1-9). When the calculation is expanded to allow substitution of the three hexoses with the twenty most commonly found sugars, the number of possible permutations rises to greater than 9 million linear and branched structures.

The availability of a large number of oligosaccharide isomers makes possible the evolution of many receptor-oligosaccharide pairs that interact in a highly specific manner. At least in part because of this large number of different isomeric permutations, carbohydrates play a significant role in a wide variety of biological interactions. For example, carbohydrates function as recognition elements that result in binding of leukocytes and other cells to their respective ligands. Carbohydrates can also serve as receptors for infectious agents, and are involved in self-recognition. Carbohydrates are often involved in signaling mechanisms.

Increased understanding of the role of carbohydrates in such biological processes has resulted in great demand for methods by which to synthesize desired carbohydrate structures. The great number of potential linkages that carbohydrates can form, while essential for the biological function of the carbohydrates, greatly complicates the synthesis of carbohydrates. For this reason, glycosyltransferases and their role in enzyme-catalyzed synthesis of carbohydrates are presently being extensively studied. Glycosyltransferases exhibit high specificity and are useful in forming carbohydrate structures of defined sequence and linkage. The use of glycosyltransferases for enzymatic synthesis of carbohydrate offers significant advantages over chemical methods due to the virtually complete stereoselectivity and linkage specificity offered by the enzymes (see, e.g., Ito et al. (1993) Pure Appl. Chem. 65: 753, and U.S. Pat. Nos. 5,352,670, and 5,374,541). Consequently, glycosyltransferases are increasingly used as enzymatic catalysts in the synthesis of a number of carbohydrates used for therapeutic and other purposes.

The commercial-scale production of carbohydrate compounds is, however, often complicated by the cost and difficulty in obtaining reactants that are used in the enzymatic and chemical synthesis of the carbohydrates. In particular, nucleotide sugars that are used as substrates for many glycosyltransferases are expensive or difficult to obtain. In addition, to make oligosaccharides for which synthesis requires more than one glycosyltransferase, the need to obtain and purify multiple glycosyltransferases can greatly increase the cost and complexity of synthesizing the oligosaccharide.

Recently, the use of cell-based systems for oligosaccharide synthesis has been described. Endo et al. ((1999) Carbohydrate Res. 316: 179-183; see also, Koizumi et al. (1998) Nature Biotechnology 16: 847-850) describe the use of a coupling of a combination of different cell types, each producing a different glycosyltransferase nucleotide sugar, to produce N-acetyllactosamine. These methods, however, require multiple cell types for each reaction, one to produce the transferase and the other to produce the nucleotide sugar.

Improved methods for enzymatic synthesis of carbohydrate compounds, and precursors used in these syntheses, would advance the production of a number of beneficial compounds. The present invention fulfills these and other needs.

### BRIEF DESCRIPTION OF THE DRAWINGS

FIG. 1A and FIG. 1B show examples in which a cell that expresses a single exogenous glycosyltransferase gene along with the corresponding nucleotide sugar is used to produce a product sugar (in this example, 3′- and 6′-sialyllactose are produced, respectively). The E. coli cell shown in FIG. 1A contains an exogenous gene that encodes a 3′-sialyltransferase, and is a particular strain that naturally produces CMP-sialic acid (CMP-SA). In FIG. 1B, the E. coli cell contains an exogenous 6′-sialyltransferase gene and, because the strain does not naturally produce sufficient amounts of CMP-sialic acid, the strain also contains an exogenous CMP-sialic acid synthetase gene. Upon expression of these enzymes and the addition lactose and other necessary reaction substrates to the reaction mixture, the desired sialyllactose is synthesized.

FIG. 2 shows an example of a cell-based enzymatic synthesis scheme in which the glycosyltransferase-expressing cell produces multiple nucleotide sugars. Because multiple nucleotide sugars are produced by the cells, the cells can be engineered to express multiple exogenous glycosyltransferases. Therefore, products that require multiple glycosidic linkages can be synthesized using a single organism. In this particular example, exogenous genes that encode two different glycosyltransferases, GlcNAc transferase and galactosyltransferase, are introduced into E. coli, which naturally produces the respective nucleotide sugar donors for these two enzymes, UDP-GlcNAc and UDP-Gal. Upon expression of the two glycosyltransferases, an acceptor saccharide and other required reactants are added to the cells in order to produce the product sugar, lacto-N-neotetraose (LNnT).

FIG. 3 illustrates a N-acetyl-glucosamine transferase cycle, as described in U.S. Pat. No. 5,922,577.

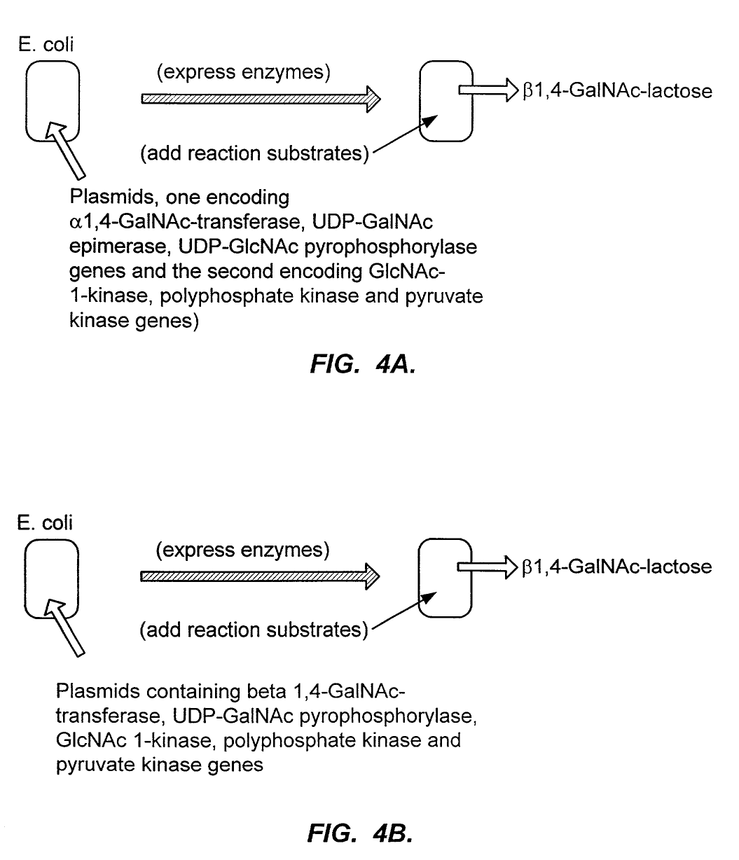

FIG. 4A and FIG. 4B show two examples of approaches in which the glycosyltransferase-expressing cell does not produce sufficient amounts of the corresponding nucleotide sugar or nucleotide. This is overcome by introducing into the cell genes that code for some or all of the enzymes of the sugar nucleotide regeneration cycle. The particular example shown involves producing GalNAc-β1,4-lactose using E. coli cells that express a β1,4 GalNAc transferase which is encoded by an exogenous gene. Because the E. coli cells do not produce sufficient amounts of the UDP-GalNAc nucleotide sugar donor or UTP, enzymes for the UDP-GalNAc cycle (shown in FIG. 3) are introduced into the cells in addition to the GalNAc transferase gene. In FIG. 4A, the system for producing UDP-GalNAc in the E. coli cells includes UDP-GaINAc epimerase, UDP-GlcNAc pyrophosphorylase, GlcNAc-1-kinase, polyphosphate kinase and pyruvate kinase. FIG. 4B shows the use of an alternative pathway for biosynthesis of UDP-GalNAc which involves the enzymes UDP-GalNAc pyrophosphorylase, GlcNAc-1-kinase, polyphosphate kinase, and pyruvate kinase. Genes that encode each of these enzymes are introduced into the E. coli cells along with the gene for the GaINAc transferase. The enzymes are expressed, after which the reaction substrates (including lactose as an acceptor) are added.

FIG. 5 shows a diagram of the enzymatic cycle for the production of PAPS, which serves as a donor for sulfotransferases. This figure is from U.S. Pat. No. 5,919,673, which provides a detailed description of the PAPS cycle. Briefly, the PAPS cycle provides a single-pot reaction system in which phosphorylated adenosine-containing moieties (AMP, ADP, ATP, APS, PAPS, and PAP) are recycled while the sulfotransferase catalyzes transfer of the sulfate group from PAPS to the acceptor moiety. In the Figure, “PEP” refers to phosphoenolpyruvate and “Pyr” refers to pyruvate.

FIGS. 6A and 6B show schematics of two examples in which two types of organisms are used to produce the nucleotide sugar. In each case, one cell type (Corynebacterium) produces a nucleotide, and the other cell type catalyzes the addition of a sugar to the nucleotide to form the nucleotide sugar. The second cell type also expresses the corresponding glycosyltransferase, which is encoded by an exogenous gene. In FIG. 6A, the desired reaction product is α-1,3-Gal-LacNAc. The reaction mixture contains Corynebacterium or yeast, for example, which naturally synthesize UTP from UDP. The UTP is activated to form UDP-galactose by the second cell type, which includes exogenous genes that encode the remaining enzymes of the GlcNAc cycle (i.e., UDP-Gal 4′ epimerase, UDP-Glc pyrophosphorylase, hexokinase and phosphoglucomutase). Also present in the second cell type is an exogenous gene that encodes α1,3-Gal transferase. The UTP that is produced by Corynebacterium or yeast enters the E. coli cells and is converted by the cycle enzymes into UDP-Gal, which then serves as a donor for galactosyltransferase-mediated transfer to a LacNAc acceptor which is also present in the reaction mixture. This reaction releases UDP, which is recycled by passing into the Corynebacterium or yeast, where it is phosphorylated to UTP. The scheme shown in FIG. 6B is useful for producing 3′-sialyllactose. Corynebacterium or yeast is again used to produce the nucleotide required for the nucleotide sugar, with the cells being engineered to produce CTP by the introduction of an exogenous gene that encodes CMP-synthetase. The E. coli cells express enzymes that are involved in the synthesis of CMP-sialic acid from CTP. In this case, the CMP-sialic acid synthetase is expressed as a fusion protein with the 3′-sialyltransferase. GIcNAc epimerase and NeuAc aldolase enzymes are also produced. This pathway converts CTP to CMP-sialic acid, which then serves as a donor for transfer of sialic acid to the lactose acceptor moiety.

FIG. 7A-D illustrate an example of a reaction scheme that employs cells that are engineered to express enzymes of the regenerating system for the active sulfating agent PAPS. These cells, when used in conjunction with a sulfotransferase, can produce sulfated sugars. The specific example shown involves the use of tobacco cells for the production of heparin or heparan. Tobacco cells, which do not naturally produce sufficient amounts of PAPS for large-scale syntheses, are engineered to contain the PAPS cycle enzymes, as well as the 3′-sulfotransferase, 6′-sulfotransferase, 2′-sulfotransferase, iduronyl-epimerase, and iduronyl-N-sulfotransferase genes. In FIG. 7A, purified K5 polysaccharide is used as the acceptor, with the resulting product being heparin sulfate. FIG. 7B is a variation on this scheme, with the K5 polysaccharide being produced by a second cell type that is included in the reaction mixture, rather than being provided in isolated form. FIG. 7C shows another variation in which heparin core polysaccharide is produced by yeast or bacterial cells that produce UDP-GlcNAc and UDP-Glc, which serve as donor sugars for the exogenous β1,4-GlcNAc transferase, β1,4-glucuronyltransferase and UDP-Glc dehydrogenases. The resulting heparin core polysaccharide produced by the yeast or bacterial cells is then added, either simultaneously or sequentially, to the cells that produce PAPS. FIG. 7D shows yet another variation of this scheme for heparin sulfate production. The Aspergillus niger that expresses the PAPS cycle enzymes does not produce sufficient amounts of ATP or PAPS cycle reagents. To produce sufficient ATP, a third cell type (e.g., yeast) is included in the reaction mixture.

FIG. 8 illustrates an example of a cell-based reaction system for a three-step enzymatic synthesis of ganglioside GM₂ (GalNAcβ4(Neu5Acα3)Galβ4GlcCer) from a lyso-glucosylceramide or lactosylceramide acceptor. This reaction involves the galactosylation of the acceptor, followed by the addition of a GalNAc residue to the galactose. Finally, sialic acid is attached. In the illustrated reaction scheme, cells that naturally produce UDP-GalNAc and UDP-Gal are engineered to express β1,4-GalNAc transferase and β1,4Gal transferase from exogenous genes. These cells are introduced into a reaction mixture along with a second cell type (e.g., Corynebacterium or yeast) that produces naturally CTP and contains exogenous genes that encode enzymes necessary for synthesis of CMP-sialic acid. The exogenous genes for CMP-sialic acid synthesis include CMP-sialic acid synthetase, GlcNAc epimerase, NeuAc aldolase, and CMP-synthetase. The second cell type also expresses an α2,3-sialyltransferase encoded by an exogenous gene.

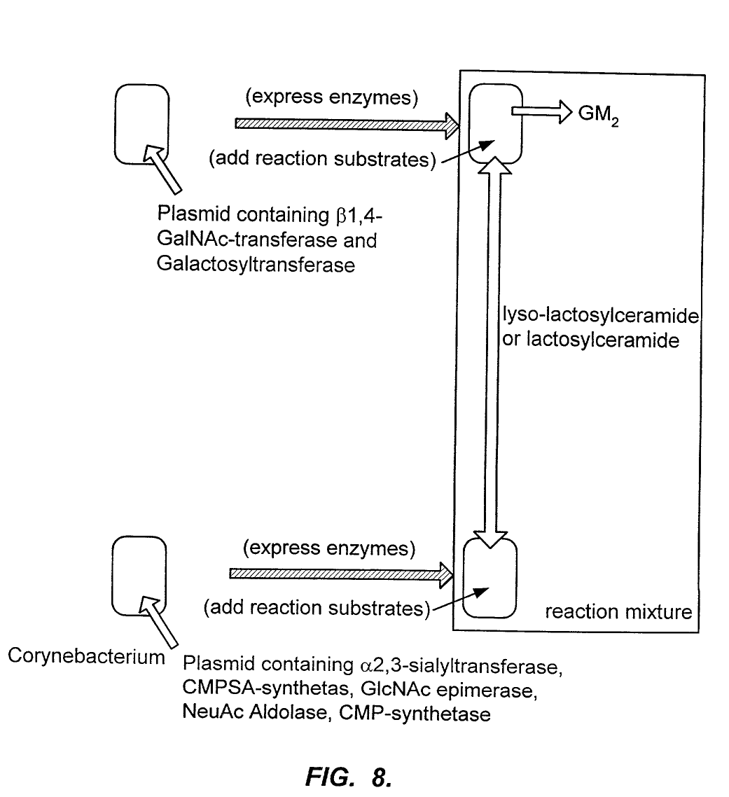

FIG. 9 shows one example of a cell-based reaction scheme for the enzymatic synthesis of the ganglioside GD₂ (GalNAcβ4(Neu5Acα8Neu5Acα3)-Galβ4GlcCer). The process involves four enzymatic reaction steps to produce GD₂ from a lyso-glucosylceramide or lactosylceramide acceptor. As in FIG. 8, two cell types are used, one that produces an exogenous α2,3-sialyltransferase and an exogenous α2,8-sialyltransferase, as well as the sugar nucleotide CMP-sialic acid, and another cell type that contains exogenous genes that encode a β1,4-GalNAc transferase and a β1,4-Gal transferase. This cell type naturally produces the respective nucleotide sugar donors for these two glycosyltransferases, UDP-GalNAc and UDP-Gal. Upon addition of the acceptor molecule and other necessary reaction substrates, GD₂ is produced by the sequential reaction of each of the four enzymes.

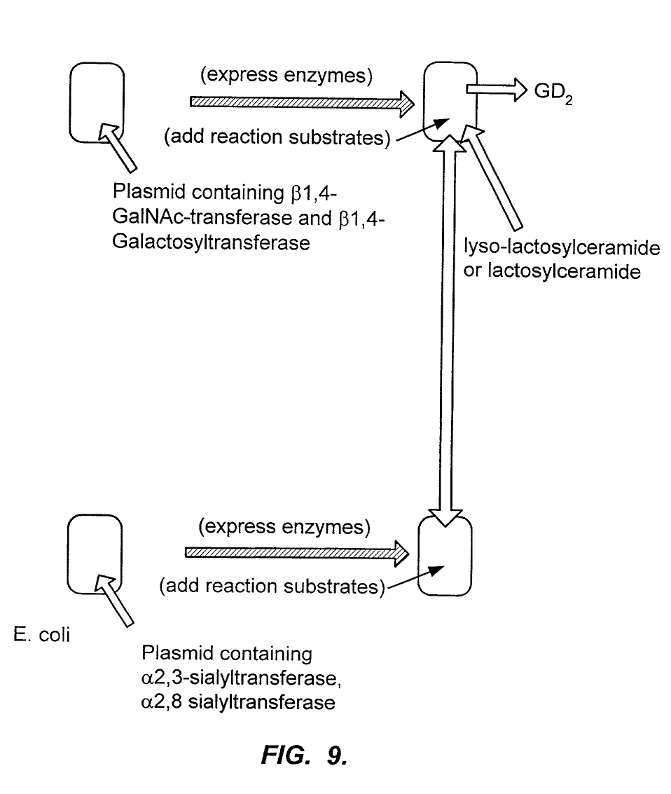

FIG. 10 shows an example of a cell-based reaction scheme for the synthesis of 3′-sialyl-LNnT (LSTd). Two cell types are used. The first cell type, E. coli in this example, naturally produces the nucleotide sugars UDP-GlcNAc and UDP-Gal. Exogenous genes that encode β1,3-GlcNAc transferase and β1,4-Gal transferase are introduced into the cells. The second cell type contains an exogenous gene that encodes an α2,3-sialyltransferase, and also produces the required sugar donor, CMP-sialic acid. Introducing both cell types into a reaction mixture along with lactose as an acceptor and other required reactants results in the production of LSTd.

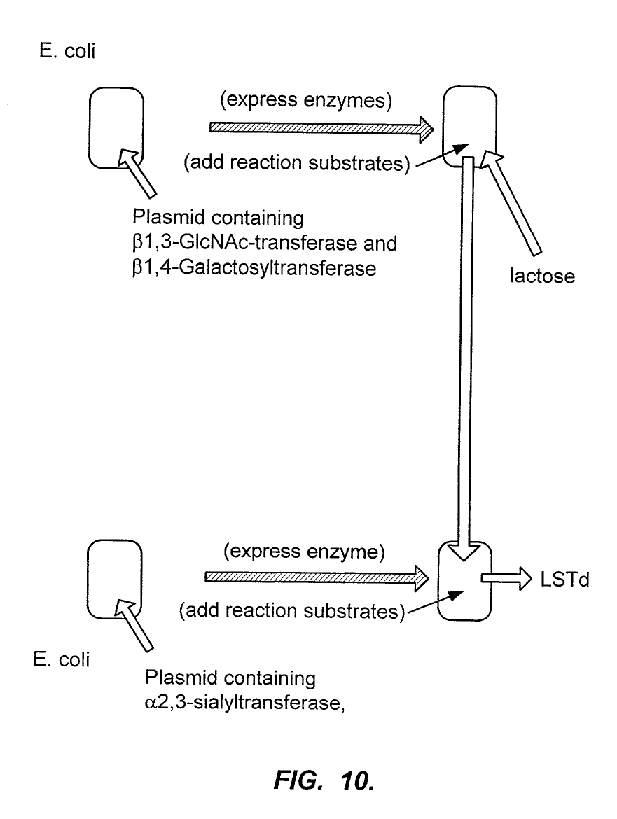

FIG. 11A and FIG. 11B show examples of cell-based reaction schemes for producing product sugars that terminate with a Galα1,3Galβ1,4GlcNAc-moiety. In FIG. 11A, Cells that naturally produce UDP-galactose are modified to express exogenous genes that encode an (α1,3-galactosyltransferase and a β1,4-galactosyltransferase. Upon addition of the acceptor sugar GlcNAc-R, the two galactosyltransferases act in sequence to add first a β1,4-linked galactose and then an α1,3-linked terminal galactose. FIG. 11B shows a variation in which the cell type produces sufficient UTP for a large-scale synthesis, but does not produce sufficient UDP-galactose. To rectify this situation, genes that encode enzymes involved in UDP-Gal synthesis (e.g., UDP-Gal 4′-epimerase and UDP-GlcNAc pyrophosphorylase) are introduced into the cells. These enzymes catalyze the conversion of the reactant glucose-1-phosphate to UDP-Gal, which in turn serves as a sugar donor for each of the two glycosyltransferases. Again, two Gal residues are linked to the GlcNAc-R acceptor saccharide.

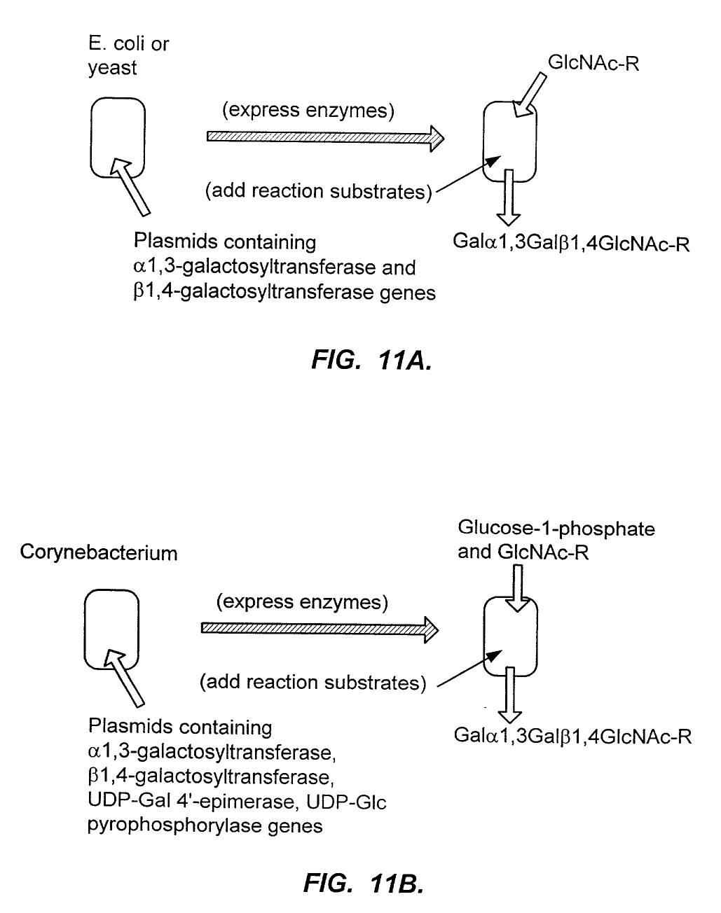

### SUMMARY OF THE INVENTION

The present invention provides reaction mixtures for producing a product saccharide. The reaction mixtures typically include an acceptor saccharide and a first type of plant or microorganism cells that each produce: a) a nucleotide sugar, and b) a recombinant glycosyltransferase that catalyzes the transfer of the sugar from the nucleotide sugar to the acceptor saccharide to form the product saccharide. In some embodiments, the reaction mixture also includes a second type of cells that each produce a) a second nucleotide sugar, and b) a second recombinant glycosyltransferase that catalyzes the transfer of the sugar from the second nucleotide sugar to the soluble oligosaccharide to form a second soluble oligosaccharide

In another embodiment, the invention provides cells that produce a product saccharide. The cells typically include a) a recombinant gene that encodes a glycosyltransferase; b) an enzymatic system for forming a nucleotide sugar which is a substrate for the glycosyltransferase; and c) an exogenous saccharide acceptor moiety. The glycosyltransferase catalyzes the transfer of a sugar from the nucleotide sugar to the acceptor moiety to produce the oligosaccharide of interest.

The invention also provides methods of producing a product saccharide. These methods involve contacting a microorganism or plant cell with an acceptor saccharide, wherein the cell includes: a) an enzymatic system for producing a nucleotide sugar; and b) a recombinant glycosyltransferase which catalyzes the transfer of a sugar from the nucleotide sugar to the acceptor saccharide to produce the product saccharide.

### DETAILED DESCRIPTION

Definitions

The cells, reaction mixtures, and methods of the invention are useful for producing a product sugar, generally by transferring a monosaccharide or a sulfate group from a donor substrate to an acceptor molecule. The addition generally takes place at the non-reducing end of an oligosaccharide, polysaccharide (e.g., heparin, carragenin, and the like) or a carbohydrate moiety on a biomolecule. Biomolecules as defined here include but are not limited to biologically significant molecules such as carbohydrates, proteins (e.g., glycoproteins), and lipids (e.g., glycolipids, phospholipids, sphingolipids and gangliosides).

The following abbreviations are used herein:

Ara=arabinosyl;

Fru=fructosyl;

Fuc=fucosyl;

Gal=galactosyl;

GalNAc=N-acetylgalactosaminyl;

Glc=glucosyl;

GlcNAc=N-acetylglucosaminyl;

Man=mannosyl; and

NeuAc=sialyl (N-acetylneuraminyl).

Typically, sialic acid is 5-N-acetylneuraminic acid, (NeuAc) or 5-N-glycolylneuraminic acid (NeuGc). Other sialic acids may be used in their place, however. For a review of different forms of sialic acid suitable in the present invention see, Schauer, Methods in Enzymology, 50: 64-89 (1987), and Schaur, Advances in Carbohydrate Chemistry and Biochemistry, 40: 131-234.

Donor substrates for glycosyltransferases are activated nucleotide sugars. Such activated sugars generally consist of uridine and guanosine diphosphates, and cytidine monophosphate derivatives of the sugars in which the nucleoside diphosphate or monophosphate serves as a leaving group. Bacterial, plant, and fungal systems can sometimes use other activated nucleotide sugars.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

All oligosaccharides described herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3, 2→3, or (2,3). Each saccharide is a pyranose or furanose.

Much of the nomenclature and general laboratory procedures required in this application can be found in Sambrook, et al., Molecular Cloning: A Laboratory Manual (2nd Ed.), Vol. 1-3, Cold Spring Harbor Laboratory, Cold Spring Harbor, N.Y. 1989. The manual is hereinafter referred to as “Sambrook et al.”

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The term “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant nucleic acid” refers to a nucleic acid that was artificially constructed (e.g., formed by linking two naturally-occurring or synthetic nucleic acid fragments). This term also applies to nucleic acids that are produced by replication or transcription of a nucleic acid that was artificially constructed. A “recombinant polypeptide” is expressed by transcription of a recombinant nucleic acid, followed by translation of the resulting transcript.

A “heterologous polynucleotide” or a “heterologous nucleic acid”, as used herein, is one that originates from a source foreign to the particular host cell, or, if from the same source, is modified from its original form. Thus, a heterologous glycosyltransferase gene in a prokaryotic host cell includes a glycosyltransferase gene that is endogenous to the particular host cell but has been modified. Modification of the heterologous sequence may occur, e.g., by treating the DNA with a restriction enzyme to generate a DNA fragment that is capable of being operably linked to a promoter. Techniques such as site-directed mutagenesis are also useful for modifying a heterologous sequence.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

A “fusion glycosyltransferase polypeptide” of the invention is glycosyltransferase fusion polypeptide that contains a glycosyltransferase catalytic domain and a second catalytic domain from an accessory enzyme (e.g., a CMP-Neu5Ac synthetase or a UDP-Glucose 4′ epimerase (galE)) and is capable of catalyzing the transfer of an oligosaccharide residue from a donor substrate (e.g., CMP-NeuAc or UDP-Gal) to an acceptor molecule. Typically, such polypeptides will be substantially similar to the exemplified proteins disclosed here.

An “accessory enzyme,” as referred to herein, is an enzyme that is involved in catalyzing a reaction that, for example, forms a substrate for a glycosyltransferase. An accessory enzyme can, for example, catalyze the formation of a nucleotide sugar that is used as a donor moiety by a glycosyltransferase. An accessory enzyme can also be one that is used in the generation of a nucleotide triphosphate required for formation of a nucleotide sugar, or in the generation of the sugar which is incorporated into the nucleotide sugar.

A “catalytic domain” refers to a portion of an enzyme that is sufficient to catalyze an enzymatic reaction that is normally carried out by the enzyme. For example, a catalytic domain of a sialyltransferase will include a sufficient portion of the sialyltransferase to transfer a sialic acid residue from a donor to an acceptor saccharide. A catalytic domain can include an entire enzyme, a subsequence thereof, or can include additional amino acid sequences that are not attached to the enzyme or subsequence as found in nature.

The term “isolated” is meant to refer to material which is substantially or essentially free from components which interfere with the activity of an enzyme. For nucleic acids of the invention, the term “isolated” refers to material that is substantially or essentially free from components which normally accompany the nucleic acid as found in its native state. Typically, isolated proteins or nucleic acids of the invention are at least about 80% pure, usually at least about 90%, and preferably at least about 95% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W,T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

The phrases “specifically binds to a protein” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind preferentially to a particular protein and do not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W. H. Freeman and Company.

Description of the Preferred Embodiments

The present invention provides cell-based methods for enzymatically synthesizing product sugars. In particular, preferred embodiments of the methods utilize sugar nucleotide recycling as well as a glycosyltransferase to manufacture product sugars, including oligosaccharides, polysaccharides, lipooligosaccharides, gangliosides, lipopolysaccharides, glycoproteins. Unlike previously available methods for saccharide synthesis, the present invention combines these components into a single recombinant organism or cell.

The product sugars are produced by contacting an acceptor saccharide with at least one cell type that contains: a) an enzymatic system for producing a nucleotide sugar, and b) a recombinant glycosyltransferase which catalyzes the transfer of a sugar from the nucleotide sugar to the acceptor saccharide to produce the product sugar. Also provided by the invention are recombinant cells that can be used in the methods, as well as reaction mixtures that include the recombinant cells and are useful for producing the product sugars. The recombinant cells provided by the invention typically contain: a) a heterologous gene that encodes a glycosyltransferase; b) an enzymatic system for forming a nucleotide sugar which is a donor substrate for the glycosyltransferase; and c) an exogenous saccharide acceptor moiety. The glycosyltransferase catalyzes the transfer of the sugar moiety from the nucleotide sugar to the acceptor, thus forming the product saccharide.

The cell-based reaction mixtures and methods of the invention provide significant advantages over previously available methods for enzymatic synthesis of oligosaccharides. Nucleotide sugars, which serve as donor substrates for glycosyltransferases, are often expensive to obtain. Thus, one advantage of the present invention is that the need to supply activated nucleotide sugars is eliminated. The organisms of the invention can continuously produce the sugar nucleotide and/or the nucleotide to which the sugar is attached. Recycling of the spent nucleotide produced from the transfer of the sugar from the sugar nucleotide during product formation can also occur because the organism contains the enzymatic processes to reform either the sugar nucleotide or nucleotide. The recombinant glycosyltransferase enzymes are also present, so the continuous production of product can occur starting from low cost raw materials.

Thus, through the use of cells that produce not only a particular glycosyltransferase, but also can synthesize from inexpensive reactants the nucleotide sugar donor for the glycosyltransferase, one can achieve highly efficient, rapid, and relatively low cost synthesis of a desired product saccharide. Saccharides produced using the methods of the invention find many uses, including, for example, diagnostic and therapeutic uses, foodstuffs, and the like.

A. Recombinant Cells that Express Glycosyltransferases and Nucleotide Sugar-Synthesizing Enzymes

The invention provides recombinant cells that express at least one glycosyltransferase, as well as produce a nucleotide sugar that can function as a sugar donor for the glycosyltransferase. The glycosyltransferase is generally encoded by a heterologous nucleic acid. Optionally, the cells can also contain an exogenous gene that encodes an enzyme involved in the synthesis of a nucleotide sugar. This enzyme is typically part of an enzymatic system for producing the nucleotide sugar. The heterologous nucleic acids can be, for example, polynucleotides that are not endogenous to the cell, or can be a modified form of a polynucleotide that is endogenous to the cell. In some applications, the cells will contain more than one exogenous glycosyltransferase gene and/or more than one exogenous gene that encodes an enzyme involved in nucleotide sugar synthesis.

The recombinant cells of the invention are generally made by creating a polynucleotide that encodes the particular enzyme, modified as desired, placing the polynucleotide in an expression cassette under the control of a promoter and other appropriate control signals, and introducing the expression cassette into a cell. More than one of the enzymes can be expressed in the same host cells, either on the same expression vector or on more than one expression vector that is present in the cells.

1. Glycosyltransferases

The recombinant cells of the invention contain at least one heterologous gene that encodes a glycosyltransferase. Many glycosyltransferases are known, as are their polynucleotide sequences. See, e.g., “The WWW Guide To Cloned Glycosyltransferases,” (http)://www.vei.co.uk/TGN/gt.guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed in the cells of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, and oligosaccharyltransferases. These glycosyltransferases include those obtained from both eukaryotes and prokaryotes. Many mammalian glycosyltransferases have been cloned and expressed and the recombinant proteins have been characterized in terms of donor and acceptor specificity and they have also been investigated through site directed mutagenesis in attempts to define residues involved in either donor or acceptor specificity (Aoki et al. (1990) EMBO. J. 9: 3171-3178; Harduin-Lepers et al. (1995) Glycobiology 5(8): 741-758; Natsuka and Lowe (1994) Current Opinion in Structural Biology 4: 683-691; Zu et al. (1995) Biochem. Biophys. Res. Comm. 206(1): 362-369; Seto et al. (1995) Eur. J. Biochem. 234: 323-328; Seto et al. (1997) J. Biol. Chem. 272: 14133-141388).

In some embodiments, the glycosyltransferase is a fucosyltransferase. A number of fucosyltransferases are known to those of skill in the art. Briefly, fucosyltransferases include any of those enzymes which transfer L-fucose from GDP-fucose to a hydroxy position of an acceptor sugar. In some embodiments, for example, the acceptor sugar is a GlcNAc in a βGal(1→4)βGlcNAc group in an oligosaccharide glycoside. Suitable fucosyltransferases for this reaction include the known βGal(1→3,4)βGlcNAc α(1→3,4)fucosyltransferase (FTIII E.C. No. 2.4.1.65) which is obtained from human milk (see, Palcic, et al., Carbohydrate Res. 190:1-11 (1989); Prieels, et al., J. Biol. Chem. 256:10456-10463 (1981); and Nunez, et al., Can. J. Chem. 59:2086-2095 (1981)) and the βGal(1→4)βGlcNAc α(1→3)fucosyltransferases (FTIV, FTV, FTVI, and FTVII, E.C. No. 2.4.1.65) which are found in human serum. A recombinant form of βGal(1→3,4)βGlcNAc α(1→3,4)fucosyltransferase is also available (see, Dumas, et al., Bioorg. Med. Letters 1:425-428 (1991) and Kukowska-Latallo, et al., Genes and Development 4:1288-1303 (1990)). Other exemplary fucosyltransferases include α1,2 fucosyltransferase (E.C. No. 2.4.1.69). Enzymatic fucosylation may be carried out by the methods described in Mollicone, et al., Eur. J. Biochem. 191:169-176 (1990) or U.S. Pat. No. 5,374,655.

In another group of embodiments, the glycosyltransferase is a galactosyltransferase. When a galactosyltransferase is used, the reaction medium will preferably contain, in addition to the cell that contains the exogenous galactosyltransferase gene and an enzymatic system for synthesizing UDP-Gal, an oligosaccharide acceptor moiety, and a divalent metal cation. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345:229-233 (1990), bovine (GenBank j04989, Joziasse et al. (1989) J. Biol. Chem. 264:14290-14297), murine (GenBank m26925; Larsen et al. (1989) Proc. Nat'l. Acad. Sci. USA 86:8227-8231), porcine (GenBank L36152; Strahan et al (1995) Immunogenetics 41:101-105)). Another suitable α1,3 galactosyltransferase is that which is involved in synthesis of the blood group B antigen (EC 2.4.1.37, Yamamoto et al. (1990) J. Biol. Chem. 265:1146-1151 (human)). Also suitable for use in the methods and recombinant cells of the invention are α(1,4) galactosyltransferases, which include, for example, EC 2.4.1.90 (LacNAc synthetase) and EC 2.4.1.22 (lactose synthetase) (bovine (D'Agostaro et al (1989) Eur. J. Biochem. 183:211-217), human (Masri et al. (1988) Biochem. Biophys. Res. Commun. 157:657-663), murine (Nakazawa et al. (1988) J. Biochem. 104:165-168), as well as E.C. 2.4.1.38 and the ceramide galactosyltransferase (EC 2.4.1.45, Stahl et al. (1994) J. Neurosci. Res. 38:234-242). Other suitable galactosyltransferases include, for example, α1,2 galactosyltransferases (from e.g., Schizosaccharomyces pombe, Chapell et al (1994) Mol. Biol. Cell 5:519-528).

Sialyltransferases are another type of glycosyltransferase that is useful in the recombinant cells and reaction mixtures of the invention. Examples of sialyltransferases that are suitable for use in the present invention include ST3Gal III (preferably a rat ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al. (1996) Glycobiology 6: v-xiv). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem., 256:3159 (1981), Weinstein et al, J. Biol. Chem., 257:13845 (1982) and Wen et al., J. Biol. Chem., 267:21011 (1992). Another exemplary α2,3-sialyltransferase (EC 2.4.99.4) transfers sialic acid to the non-reducing terminal Gal of the disaccharide or glycoside. See, Rearick et al., J. Biol. Chem., 254:4444 (1979) and Gillespie et al., J. Biol. Chem., 267:21004 (1992). Further exemplary enzymes include Gal-β-1,4-GlcNAc α-2,6 sialyltransferase (See, Kurosawa et al. Eur. J. Biochem. 219: 375-381 (1994)).

Other glycosyltransferases that can be contained by the recombinant host cells of the invention have been described in detail, as for the sialyltransferases, galactosyltransferases, and fucosyltransferases. In particular, the glycosyltransferase can also be, for instance, glucosyltransferases, e.g., Alg8 (Stagljov et al., Proc. Natl. Acad. Sci. USA 91:5977 (1994)) or Alg5 (Heesen et al. Eur. J. Biochem. 224:71 (1994)), N-acetylgalactosaminyltransferases such as, for example, α(1,3) N-acetylgalactosaminyltransferase, β(1,4) N-acetylgalactosaminyltransferases (Nagata et al. J. Biol. Chem. 267:12082-12089(1992) and Smith et al. J. Biol Chem. 269:15162(1994)) and polypeptide N-acetylgalactosaminyltransferase (Homa et al. J. Biol Chem. 268:12609 (1993)). Suitable N-acetylglucosaminyltransferases include GnTI (2.4.1.101, Hull et al., BBRC 176:608 (1991)), GnTII, and GnTIII (Ihara et al. J. Biochem. 113:692 (1993)), GnTV (Shoreiban et al. J. Biol. Chem. 268: 15381 (1993)), O-linked N-acetylglucosaminyltransferase (Bierhuizen et al. Proc. Natl. Acad. Sci. USA 89:9326 (1992)), N-acetylglucosamine-1-phosphate transferase (Rajput et al. Biochem J.285:985 (1992), and hyaluronan synthase. Suitable mannosyltransferases include α(1,2) mannosyltransferase, α(1,3) mannosyltransferase, β(1,4) mannosyltransferase, Dol-P-Man synthase, OCh1, and Pmt1.

Prokaryotic glycosyltransferases are also useful in the recombinant cells and reaction mixtures of the invention. Such glycosyltransferases include enzymes involved in synthesis of lipooligosaccharides (LOS), which are produced by many gram negative bacteria. The LOS typically have terminal glycan sequences that mimic glycoconjugates found on the surface of human epithelial cells or in host secretions (Preston et al. (1996) Critical Reviews in Microbiology 23(3): 139-180). Such enzymes include, but are not limited to, the proteins of the rfa operons of species such as E. coli and Salmonella typhimurium, which include a β1,6 galactosyltransferase and β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an β1,2-glucosyltransferase (rfaJ)(Swiss-Prot Accession No. P27129 (E. coli) and Swiss-Prot Accession No. P19817 (S. typhimurium)), and an β1,2-N-acetylglucosaminyltransferase (rfaK)(EMBL Accession No. U00039 (E. coli). Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rhl operon of Pseudomonas aeruginosa.

Also suitable for use in the cells of the invention are glycosyltransferases that are involved in producing structures containing lacto-N-neotetraose, D-galactosyl-β-1,4-N-acetyl-D-glucosaminyl-β-1,3-D-galactosyl-β-1,4-D-glucose, and the P^{k} blood group trisaccharide sequence, D-galactosyl-α-1,4-D-galactosyl-β-1,4-D-glucose, which have been identified in the LOS of the mucosal pathogens Neisseria gonnorhoeae and N. meningitidis (Scholten et al. (1994) J. Med. Microbiol. 41: 236-243). The genes from N. meningitidis and N. gonorrhoeae that encode the glycosyltransferases involved in the biosynthesis of these structures have been identified from N. meningitidis immunotypes L3 and L1 (Jennings et al. (1995) Mol. Microbiol. 18: 729-740) and the N. gonorrhoeae mutant F62 (Gotshlich (1994) J. Exp. Med. 180: 2181-2190). In N. meningitidis, a locus consisting of three genes, lgtA, lgtB and lg E, encodes the glycosyltransferase enzymes required for addition of the last three of the sugars in the lacto-N-neotetraose chain (Wakarchuk et al. (1996) J. Biol. Chem. 271: 19166-73). Recently the enzymatic activity of the lgtB and lgtA gene product was demonstrated, providing the first direct evidence for their proposed glycosyltransferase function (Wakarchuk et al. (1996) J. Biol. Chem. 271 (45): 28271-276). In N. gonorrhoeae, there are two additional genes, lgtD which adds β-D-GalNAc to the 3 position of the terminal galactose of the lacto-N-neotetraose structure and lgtC which adds a terminal α-D-Gal to the lactose element of a truncated LOS, thus creating the p^{k} blood group antigen structure (Gotshlich (1994), supra.). In N. meningitidis, a separate immunotype L1 also expresses the P^{k} blood group antigen and has been shown to carry an lgtC gene (Jennings et al. (1995), supra.). Neisseria glycosyltransferases and associated genes are also described in U.S. Pat. No. 5,545,553 (Gotschlich). An α1,3-fucosyltransferase gene from Helicobacter pylori has also been characterized (Martin et al. (1997) J. Biol. Chem. 272: 21349-21356).

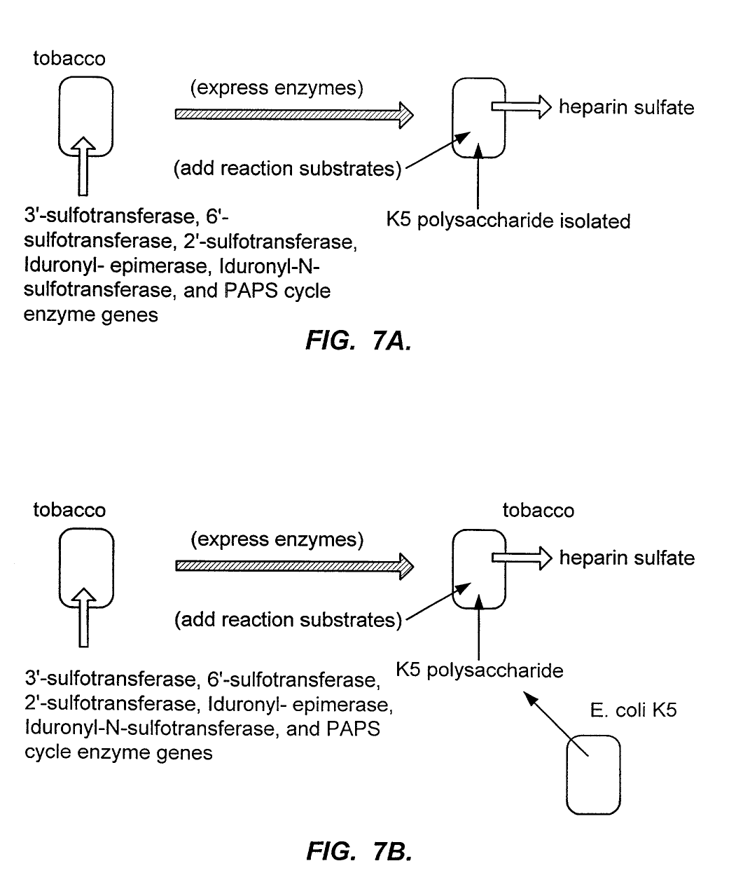

In some embodiments, the recombinant cells of the invention will contain at least one heterologous gene that encodes a sulfotransferase. Such cells also produce the active sulfating agent 3′-phosphoadenosine-5′-phosphosulfate (PAPS). Incorporation of one or more sulfotransferase genes into a cell that also produces PAPS, either naturally or through the addition of the PAPS cycle regeneration enzymes, provides one with cells that can sulfate oligosaccharides or polysaccharides (FIG. 5). Suitable sulfotransferases include, for example, chondroitin-6-sulphotransferase (chicken cDNA described by Fukuta et al. (1995) J. Biol. Chem. 270:18575-18580; GenBank Accession No. D49915), glycosaminoglycan N-acetylglucosamine N-deacetylase/N-sulphotransferase 1 (Dixon et al. (1995) Genomics 26:239-241; UL18918), and glycosaminoglycan N-acetylglucosamine N-deacetylase/N-sulphotransferase 2 (murine cDNA described in Orellana et al. (1994) J. Biol. Chem. 269:2270-2276 and Eriksson et al. (1994) J. Biol. Chem. 269:10438-10443; human cDNA described in GenBank Accession No. U2304).

Glycosyltransferase nucleic acids, and methods of obtaining such nucleic acids, are known to those of skill in the art. Glycosyltransferase nucleic acids (e.g., cDNA, genomic, or subsequences (probes)) can be cloned, or amplified by in vitro methods such as the polymerase chain reaction (PCR), the ligase chain reaction (LCR), the transcription-based amplification system (TAS), the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning: A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al. (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Nat'l. Acad. Sci USA 86: 1173; Guatelli et al. (1990) Proc. Nat'l. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

DNA that encodes glycosyltransferase proteins or subsequences, as well as DNA that encodes the enzymes involved in formation of nucleotide sugars described below, can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. In one preferred embodiment, a nucleic acid encoding a glycosyltransferase can be isolated by routine cloning methods. A nucleotide sequence of a glycosyltransferase as provided in, for example, GenBank or other sequence database can be used to provide probes that specifically hybridize to a glycosyltransferase gene in a genomic DNA sample, or to a glycosyltransferase mRNA in a total RNA sample (e.g., in a Southern or Northern blot). Once the target glycosyltransferase nucleic acid is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York).

A glycosyltransferase nucleic acid can also be cloned by detecting its expressed product by means of assays based on the physical, chemical, or immunological properties. For example, one can identify a cloned glycosyltransferase nucleic acid by the ability of a polypeptide encoded by the nucleic acid to catalyze the transfer of a monosaccharide from a donor to an acceptor moiety. In a preferred method, capillary electrophoresis is employed to detect the reaction products. This highly sensitive assay involves using either monosaccharide or disaccharide aminophenyl derivatives which are labeled with fluorescein as described in Wakarchuk et al. (1996) J. Biol. Chem. 271 (45): 28271-276. For example, to assay for a Neisseria lgtC enzyme, either FCHASE-AP-Lac or FCHASE-AP-Gal can be used, whereas for the Neisseria lgtB enzyme an appropriate reagent is FCHASE-AP-GlcNAc (Id.).

As an alternative to cloning a glycosyltransferase gene, a glycosyltransferase nucleic acid can be chemically synthesized from a known sequence that encodes a glycosyltransferase. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is often limited to sequences of about 100 bases, longer sequences may be obtained by the ligation of shorter sequences.

Alternatively, subsequences can be cloned and the appropriate subsequences cleaved using appropriate restriction enzymes. The fragments may then be ligated to produce the desired DNA sequence.

In one embodiment, glycosyltransferase nucleic acids can be cloned using DNA amplification methods such as polymerase chain reaction (PCR). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired glycosyltransferase sequence or subsequence and having terminal restriction sites. This nucleic acid can then be easily ligated into a vector containing a nucleic acid encoding the second molecule and having the appropriate corresponding restriction sites. Suitable PCR primers can be determined by one of skill in the art using the sequence information provided in GenBank or other sources. Appropriate restriction sites can also be added to the nucleic acid encoding the glycosyltransferase protein or protein subsequence by site-directed mutagenesis. The plasmid containing the glycosyltransferase-encoding nucleotide sequence or subsequence is cleaved with the appropriate restriction endonuclease and then ligated into an appropriate vector for amplification and/or expression according to standard methods.

Other physical properties of a polypeptide expressed from a particular nucleic acid can be compared to properties of known glycosyltransferases to provide another method of identifying glycosyltransferase-encoding nucleic acids. Alternatively, a putative glycosyltransferase gene can be mutated, and its role as a glycosyltransferase established by detecting a variation in the structure of an oligosaccharide normally produced by the glycosyltransferase.

In some embodiments, it may be desirable to modify the glycosyltransferase or accessory enzyme nucleic acids. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

In a preferred embodiment, the recombinant nucleic acids present in the cells of the invention are modified to include preferred codons which enhance translation of the nucleic acid in a selected organism (e.g., yeast preferred codons are substituted into a coding nucleic acid for expression in yeast).

2. Accessory Enzymes Involved in Synthesis of Nucleotide Sugars and Other Reactants

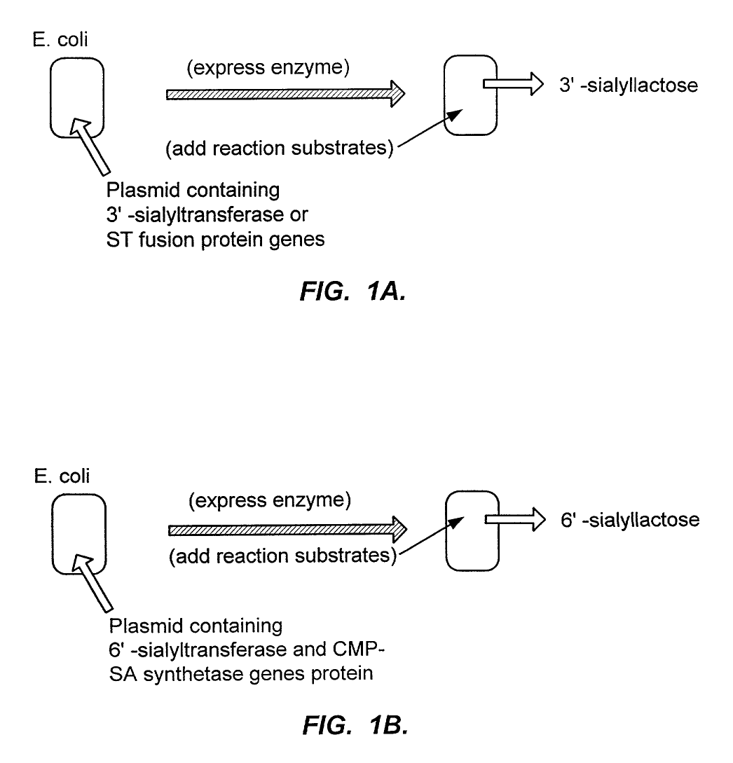

Glycosyltransferase reactions require a nucleotide sugar which serves as sugar donor. In some embodiments, the recombinant cells of the invention can naturally produce the sugar nucleotide that serves as a sugar donor for the glycosyltransferase produced by the cell, as well as the nucleotide to which the sugar molecule is attached (FIG. 1A). However, some cells do not naturally produce sufficient amounts of either or both of the nucleotide or the nucleotide sugar to produce the desired quantities of product saccharide. In such situations, the recombinant cells of the invention contain not only a heterologous gene for the glycosyltransferase, but also at least one heterologous gene that encodes an accessory enzyme (FIG. 1B).

Accessory enzymes include those enzymes that are involved in the formation of a nucleotide sugar. The accessory enzyme can be involved in attaching the sugar to a nucleotide, or can be involved in making the sugar or the nucleotide, for example. Because the organism continues to produce either the nucleotide or sugar nucleotide and the recombinant enzymes are also present, the continuous production of product can occur starting from low cost raw materials. Recycling of the spent nucleotide produced from the transfer of the sugar from the sugar nucleotide during product formation can also occur as the organism contains the enzymatic processes to reform either the sugar nucleotide or nucleotide. Accessory enzymes that are involved in synthesis of nucleotide sugars are well known to those of skill in the art. For a review of bacterial polysaccharide synthesis and gene nomenclature, see, e.g., Reeves et al., Trends Microbiol. 4: 495-503 (1996).

The enzymatic system for forming the nucleotide sugar includes, in presently preferred embodiments, an enzyme encoded by a heterologous gene. Such cells provide a means for forming a desired nucleotide sugar that is not normally produced by the cell, or is not produced at a sufficiently high level by the cell. In some instances, the enzyme encoded by the heterologous gene can convert a nucleotide or nucleotide sugar that is produced by the cell into a different nucleotide sugar that can serve as a substrate for the desired coupling reaction. In other cases, the enzyme encoded by the heterologous gene can synthesize a nucleotide sugar from other substrates (e.g., nucleotides) that are found in the cell, either endogenously or as a result of the substrate having been added to the cell. Multiple nucleotide sugar synthesis and/or conversion reactions can be achieved by using a cell that contains more than one heterologous gene that encodes an enzyme involved in nucleotide sugar synthesis.

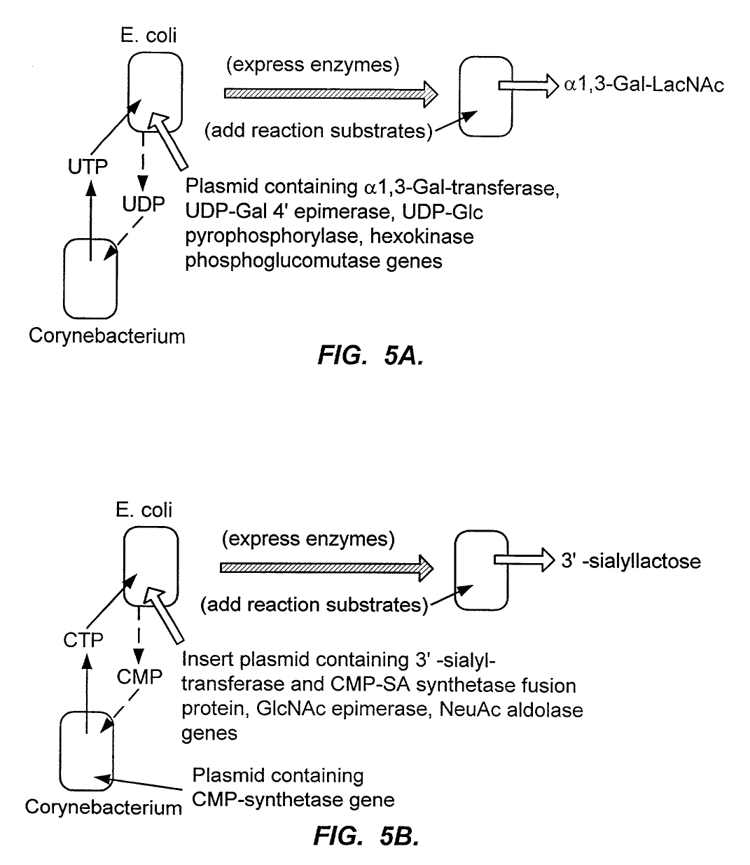

The genes encoding enzymes for an entire sugar nucleotide regeneration cycle can be introduced into an organism along with the glycosyltransferase of interest. The resulting recombinant cells can thus produce both the desired nucleotide sugar and the final product (FIG. 4A and FIG. 4B). Pathways and enzymes that are involved in synthesis of nucleotide sugars are well known to those of skill in the art. For a review of bacterial polysaccharide synthesis and gene nomenclature, see, e.g., Reeves et al. (1996) Trends Microbiol. 4: 495-503. Examples of cycle enzymes that are of use in producing various nucleotide sugars are listed in Table 1.

TABLE 1
Cycle Enzymes¹
¹Each of the cycle processes listed below requires either
a nucleotide triphosphate source or the enzymes required to
regenerate the nucleotide to its nucleotide triphosphate form.

| | |
|---|---|
| GLcNAc Cycle | GalNAc Cycle-1 |
| UDP-GLcNAc Pyrophosphorylase | UDP-GalNAc Epimerase |
| GLcNAc/GalNAc Kinase | UDP-GlcNAc Pyrophosphorylase |
| GlcNAc Transferase | GlcNAc 1-Phospho Kinase* |
| Gal Cycle-1 | *(or Hexokinase and GlcNAc |
| Gal kinase | Phosphomutase) |
| UDP-Gal Pyrophosphorylase | GlcNAc Transferase |
| Gal Transferase | GalNAc Cycle- |
| Gal Cycle-2 | UDP-GalNAc Pyrophosphorylase |
| UDP-Gal 4′-Epimerase | GlcNAc Transferase |
| UDP-Glc Pyrophosphorylase | GlcNAc/GalNAc kinase |
| Hexokinase Kinase | Man Cycle |
| Phosphoglucomutase | GDP-Man Pyrophosphorylase |
| ST Cycle | Hexokinase |
| ST fusion (sialyltransferase | Phosphomannomutase |
| fused CMP-SA synthetase)* | Man Transferase |
| *(or sialyltransferase and | Fuc Cycle-2 |
| CMP-SA synthetase) | GDP-Fuc Pyrophosphorylase |
| NeuAc Aldolase | Fucose 1-phosphokinase |
| GlcNAc Epimerase | Fucosyl Transferase |
| Fuc Cycle-1 | |
| GDP-Fuc Epimerase/reductase | |
| GDP-Fuc Dehydratase | |
| GDP-Man Pyrophosphorylase | |
| Hexokinase | |
| Phosphomannomutase | |
| Fucosyl Transferase | |

By choosing appropriate genes and placing them into a cell that contains a substrate for the enzymes encoded by the genes, one can modify one or more pathways that lead to nucleotide sugar production. The methods described above for obtaining glycosyltransferase-encoding nucleic acids are also applicable to obtaining nucleic acids that encode enzymes involved in the formation of nucleotide sugars. For example, one can use one of nucleic acids known in the art, some of which are listed below, directly or as a probe to isolate a corresponding nucleic acid from other organisms of interest.

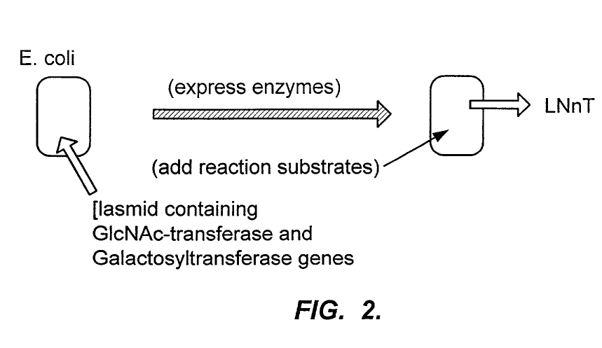

In some embodiments, the recombinant cells of the invention can produce multiple nucleotide sugars or nucleotides, thus allowing the introduction of multiple glycosyltransferases or multiple glycosyltransferase with supporting cycle enzymes, respectively, to produce the target sugar. This allows the production of multiple glycosidic linkages in a product using a single organism. For example, if the organism produces both UDP-Gal and UDP-GlcNAc, then addition of a Gal transferase and a GlcNAc transferase would allow the production two new glycosidic linkages from the same organism (FIG. 2). As another example, if an organism produces elevated levels of UTP, then by adding genes that encode enzymes for the production of UDP-Gal and UDP-GlcNAc, as well as genes that encode a Gal transferase and a GlcNAc transferase two new glycosidic linkages can be formed from a single organism. In these examples, if the transferases allow glycosidic polymerization, then long chain oligosaccharides and polysaccharides can be formed.

An illustrative example of a recombinant cell that is useful for producing a galactosylated product saccharide contains a heterologous galactosyltransferase gene. However, galactosyltransferases generally use as a galactose donor the activated nucleotide sugar UDP-Gal, which is comparatively expensive. To reduce the expense of the reaction, one can introduce into the cell (or increase the level of expression of) one or more genes that encode enzymes that are involved in the biosynthetic pathway which leads to UDP-Gal. For example, glucokinase (EC 2.7.1.12) catalyzes the phosphorylation of glucose to form Glc-6-P. Genes that encode glucokinase have been characterized (e.g., E. coli: GenBank AE000497 U00096, Blattner et al. (1997) Science 277: 1453-1474; Bacillus subtilis: GenBank Z99124, AL009126, Kunst et al. (1997) Nature 390, 249-256), and thus can be readily obtained from many organisms by, for example, hybridization or amplification. A recombinant cell that contains this gene, as well as the subsequent enzymes in the pathway as set forth below, will thus be able to form GDP-glucose from readily available glucose, which can be either produced by the organism or added to the reaction mixture.

The next step in the pathway leading to UDP-Gal is catalyzed by phosphoglucomutase (EC 5.4.2.2), which converts Glc-6-P to Glc-1-P. Again, genes encoding this enzyme have been characterized for a wide range of organisms (e.g., Agrobacterium tumefaciens: GenBank AF033856, Uttaro et al. Gene 150: 117-122 (1994) [published erratum appears in Gene (1995) 155:141-3]; Entamoeba histolytica: GenBank Y14444, Ortner et al., Mol. Biochem. Parasitol. 90, 121-129 (1997); Mesembryanthemum crystallinum: GenBank U84888; S. cerevisiae: GenBank X72016, U09499, X74823, Boles et al., Eur. J. Biochem. 220: 83-96 (1994), Fu et al., J. Bacteriol. 177 (11), 3087-3094 (1995); human: GenBank M83088 (PGM1), Whitehouse et al., Proc. Nat'l. Acad. Sci. U.S.A. 89: 411-415 (1992), Xanthomonas campestris: GenBank M83231, Koeplin et al., J. Bacteriol. 174: 191-199 (1992); Acetobacter xylinum: GenBank L24077, Brautaset et al., Microbiology 140 (Pt 5), 1183-1188 (1994); Neisseria meningitidis: GenBank U02490, Zhou et al., J. Biol. Chem. 269 (15), 11162-11169 (1994).

UDP-glucose pyrophosphorylase (EC 2.7.7.9) catalyzes the next step in the pathway, conversion of Glc-1-P to UDP-Glc. Genes encoding UDP-Glc pyrophosphorylase are described for many organisms (e.g., E. coli: GenBank M98830, Weissborn et al., J. Bacteriol. 176: 2611-2618 (1994); Cricetulus griseus: GenBank AF004368, Flores-Diaz et al., J. Biol. Chem. 272: 23784-23791 (1997); Acetobacter xylinum: GenBank M76548, Brede et al., J. Bacteriol. 173, 7042-7045 (1991); Pseudomonas aeruginosa (gal U): GenBank AJ010734, U03751; Streptococcus pneumoniae: GenBank AJ004869; Bacillus subtilis: Genbank Z22516, L12272; Soldo et al., J. Gen. Microbiol. 139 (Pt 12), 3185-3195 (1993); Solanum tuberosum: GenBank U20345, L77092, L77094, L77095, L77096, L77098, U59182, Katsube et al., J. Biochem. 108: 321-326 (1990); Hordeum vulgare (barley): GenBank X91347; Shigella flexneri: GenBank L3281 1, Sandlin et al., Infect. Immun. 63: 229-237 (1995); human: GenBank U27460, Duggleby et al., Eur. J. Biochem. 235 (1-2), 173-179 (1996); bovine: GenBank L14019, Konishi et al., J. Biochem. 114, 61-68 (1993).

Finally, UDP-Glc 4′-epimerase (UDP-Gal 4′ epimerase; EC 5.1.3.2) catalyzes the conversion of UDP-Glc to UDP-Gal. The Streptococcus thermophilus UDPgalactose 4-epimerase gene described by Poolman et al. (J. Bacteriol 172: 4037-4047 (1990)) is a particular example of a gene that is useful in the present invention. UDPglucose 4-epimerase-encoding polynucleotides of other organisms can be used in the present invention, so long polynucleotides are under the control of expression control sequences that function in E. coli or other desired host cell. Exemplary organisms that have genes encoding UDPglucose 4-epimerase include E. coli, K. pneumoniae, S. lividans, and E. stewartii, as well as Salmonella and Streptococcus species. Nucleotide sequences are known for UDP-Glc 4′-epimerases from several organisms, including Pasteurella haemolytica, GenBank U39043, Potter et al., Infect. Immun. 64 (3), 855-860 (1996); Yersinia enterocolitica, GenBank Z47767, X63827, Skurnik et al., Mol. Microbiol. 17: 575-594 (1995); Cyamopsis tetragonoloba: GenBank AJ005082; Pachysolen tannophilus: GenBank X68593, Skrzypek et al., Gene 140 (1), 127-129 (1994); Azospirillum brasilense: GenBank Z25478, De Troch et al., Gene 144 (1), 143-144 (1994); Arabidopsis thaliana: GenBank Z54214, Dormann et al., Arch. Biochem. Biophys. 327: 27-34 (1996); Bacillus subtilis: GenBank X99339, Schrogel et al., FEMS Microbiol. Lett. 145: 341-348 (1996); Rhizobium meliloti: GenBank X58126 S81948, Buendia et al., Mol. Biol. 5: 1519-1530 (1991); Rhizobium leguminosarum: GenBank X96507; Erwinia amylovora: GenBank X76172, Metzger et al., J. Bacteriol. 176: 450-459 (1994); S. cerevisiae: GenBank X81324 (cluster of epimerase and UDP-glucose pyrophosphorylase), Schaaff-Gerstenschlager, Yeast 11: 79-83 (1995); Neisseria meningitidis: GenBank U19895, L20495, Lee et al., Infect. Immun. 63: 2508-2515 (1995), Jennings et al., Mol. Microbiol. 10: 361-369 (1993); and Pisum sativum: GenBank U31544.

Often, genes encoding enzymes that make up a pathway involved in synthesizing nucleotide sugars are found in a single operon or region of chromosomal DNA. For example, the Xanthomonas campestris phosphoglucomutase, phosphomannomutase, (xanA), phosphomannose isomerase, and GDP-mannose pyrophosphorylase (xanB) genes are found on a single contiguous nucleic acid fragment (Koeplin et al., J. Bacteriol. 174, 191-199 (1992)). Klebsiella pneumoniae galactokinase, galactose-1-phosphate uridyltransferase, and UDP-galactose 4′-epimerase are also found in a single operon (Peng et al. (1992) J. Biochem. 112: 604-608). Many other examples are described in the references cited herein.

An alternative way to construct a cell that makes UDP-Gal is to introduce into the cell a gene that encodes UDP-Gal pyrophosphorylase (galactose-1-phosphate uridyltransferase), which converts Gal-1-P to UDP-Gal. Genes that encode UDP-Gal pyrophosphorylase have been characterized for several organisms, including, for example, Rattus norvegicus: GenBank L05541, Heidenreich et al., DNA Seq. 3: 311-318 (1993); Lactobacillus casei: GenBank AF005933 (cluster of galactokinase (galK), UDP-galactose 4-epimerase (galE), galactose 1-phosphate-uridyltransferase (galT)), Bettenbrock et al., Appl. Environ. Microbiol. 64: 2013-2019 (1998); E. coli: GenBank X06226 (gale and galT for UDP-galactose-4-epimerase and galactose-1-P uridyltransferase), Lemaire et al., Nucleic Acids Res. 14: 7705-7711 (1986)); B. subtilis: GenBank Z99123 AL009126; Neisseria gonorrhoeae: GenBank Z50023, Ullrich et al., J. Bacteriol. 177: 6902-6909 (1995); Haemophilus influenzae: GenBank X65934 (cluster of galactose-1-phosphate uridyltransferase, galactokinase, mutarotase and galactose repressor), Maskell et al., Mol. Microbiol. 6: 3051-3063 (1992), GenBank M12348 and M12999, Tajima et al., Yeast 1: 67-77 (1985)); S. cerevisiae: GenBank X81324, Schaaff-Gerstenschlager et al., Yeast 11: 79-83 (1995); Mus musculus: GenBank U41282; human: GenBank M96264, MI 8731, Leslie et al., Genomics 14: 474-480 (1992), Reichardt et al., Mol. Biol. Med. 5: 107-122 (1988); Streptomyces lividans: M18953 (galactose 1-phosphate uridyltransferase, UDP-galactose 4-epimerase, and galactokinase), Adams et al., J. Bacteriol. 170: 203-212 (1988).

UDP-GlcNAc 4′ epimerase (UDP-GalNAc 4′-epimerase)(EC 5.1.3.7), which catalyzes the conversion of UDP-GlcNAc to UDP-GalNAc, and the reverse reaction, is also suitable for use in the recombinant cells of the invention. Several loci that encode this enzyme are described above. See also, U.S. Pat. No. 5,516,665.

Another example of a recombinant cell provided by the invention is used for producing a fucosylated product saccharide. The donor nucleotide sugar for fucosyltransferases is GDP-fucose, which is relatively expensive to produce. To reduce the cost of producing the fucosylated oligosaccharide, the invention provides cells that can convert the relatively inexpensive GDP-mannose into GDP-fucose. These cells contain at least one exogenous gene that encodes a GDP-mannose dehydratase, a GDP-4-keto-6-deoxy-D-mannose 3,5-epimerase, or a GDP-4-keto-6-deoxy-L-glucose 4-reductase. Cells that contain each of these enzyme activities can convert GDP-mannose into GDP-fucose. The introduction of a fucosyltransferase into the cell results in a cell that can fucosylate an oligosaccharide acceptor using GDP-mannose, rather than GDP-fucose, as the donor activated sugar.

The nucleotide sequence of an E. coli gene cluster that encodes GDP-fucose-synthesizing enzymes is described by Stevenson et al. (1996) J. Bacteriol. 178: 4885-4893; GenBank Accession No. U38473). This gene cluster had been reported to include an open reading frame for GDP-mannose dehydratase (nucleotides 8633-9754; Stevenson et al., supra.). It was recently discovered that this gene cluster also contains an open reading frame that encodes an enzyme that has both 3,5 epimerization and 4-reductase activities (see, commonly assigned U.S. Provisional Patent Application No. 60/071,076, filed Jan. 15, 1998), and thus is capable of converting the product of the GDP-mannose dehydratase reaction (GDP-4-keto-6-deoxymannose) to GDP-fucose. This ORF, which is designated YEF B, is found between nucleotides 9757-10722. Prior to this discovery that YEF B encodes an enzyme having two activities, it was not known whether one or two enzymes were required for conversion of GDP-4-keto-6-deoxymannose to GDP-fucose. The nucleotide sequence of a gene encoding the human Fx enzyme is found in GenBank Accession No. U58766.

The recombinant cells can also include a gene that encodes GDP-Man pyrophosphorylase (EC 2.7.7.22), which converts Man-1-P to GDP-Man. When present along with an enzyme such as those described above which catalyze the conversion of GDP-Man to GDP-Fuc, such cells can synthesize GDP-Fuc starting from the relatively inexpensive Man-1-P. Suitable genes are known from many organisms, including E. coli: GenBank U13629, AB010294, D43637 D13231, Bastin et al., Gene 164: 17-23 (1995), Sugiyama et al., J. Bacteriol. 180: 2775-2778 (1998), Sugiyama et al., Microbiology 140 (Pt 1): 59-71 (1994), Kido et al., J. Bacteriol. 177: 2178-2187 (1995); Klebsiella pneumoniae: GenBank AB010296, AB010295, Sugiyama et al., J. Bacteriol. 180: 2775-2778 (1998); Salmonella enterica: GenBank X56793 M29713, Stevenson et al., J. Bacteriol. 178: 4885-4893 (1996).

The cells of the invention for fucosylating a saccharide acceptor can also utilize enzymes that provide a minor or “scavenge” pathway for GDP-fucose formation. In this pathway, free fucose is phosphorylated by fucokinase to form fucose 1-phosphate, which, along with guanosine 5′-triphosphate (GTP), is used by GDP-fucose pyrophosphorylase to form GDP-fucose (Ginsburg et al., J. Biol. Chem., 236: 2389-2393 (1961) and Reitman, J. Biol. Chem., 255: 9900-9906 (1980)). GDP-fucose pyrophosphorylase-encoding nucleic acids are described in copending, commonly assigned U.S. patent application Ser. No. 08/826,964, filed Apr. 9, 1997. Fucokinase-encoding nucleic acids are described for, e.g., Haemophilus influenzae (Fleischmann et al. (1995) Science 269:496-512) and E. coli (Lu and Lin (1989) Nucleic Acids Res. 17: 4883-4884).

Other pyrophosphorylases are known that convert a sugar phosphate into a nucleotide sugar. For example, UDP-GalNAc pyrophosphorylase catalyzes the conversion of GalNAc to UDP-GalNAc. UDP-GlcNAc pyrophosphorylase (EC 2.7.7.23) converts GlcNAc-1-P to UDP-GlcNAc (B. subtilis: GenBank Z99104 AL009126, Kunst et al., supra.; Candida albicans: GenBank AB011003, Mio et al., J. Biol. Chem. 273 (23), 14392-14397 (1998); Saccharomyces cerevisiae: GenBank AB011272, Mio et al., supra.; human: GenBank AB011004, Mio et al., supra.).

To obtain recombinant cells of the invention that are useful for sialylation reactions, one can introduce a gene that encodes an enzyme that encodes CMP-sialic acid synthetase (EC 2.7.7.43, CMP-N-acetylneuraminic acid synthetase). Such genes are available from, for example, Mus musculus (GenBank AJ006215, Munster et al., Proc. Natl. Acad. Sci. U.S.A. 95: 9140-9145 (1998)), rat (Rodriguez-Aparicio et al. (1992) J. Biol. Chem. 267: 9257-63), Haemophilus ducreyi (Tullius et al. (1996) J. Biol. Chem. 271: 15373-80), Neisseria meningitidis (Ganguli et al. (1994) J. Bacteriol. 176: 4583-9), group B streptococci (Haft et al. (1994) J. Bacteriol. 176: 7372-4), and E. coli (GenBank J05023, Zapata et al. (1989) J. Biol. Chem. 264: 14769-14774).

The isolation of polynucleotides that encode nucleotide sugar synthetic enzymes can be performed by a number of techniques well known to those skilled in the art. For instance, oligonucleotide probes that selectively hybridize to the a particular gene described herein can be used to identify the desired gene in DNA isolated from another organism. The use of such hybridization techniques for identifying homologous genes is well known in the art are otherwise as described above.

In additional embodiments, the recombinant cells of the invention produce a nucleotide sugar at an elevated level compared to a wild-type cell, and/or a nucleotide sugar produced by the cell is diverted from, for example, production of a polysaccharide to production of a desired product saccharide. For example, Azobacter vinelandii and Pseudomonas aeruginosa produce relatively large amounts of GDP-Man, the majority of which is used in the synthesis of the polysaccharide alginate. By disrupting the ability of the cells to produce alginate, one can obtain cells that produce increased levels of GDP-Man. Alginate synthesis in Pseudomonas and Azobacter involves GDP-mannose dehydrogenase, which converts GDP-Man to GDP-mannuronic acid, which is a direct precursor of alginate (Tatnell et al. (1994) Microbiol. 140: 1745-1754; Tatnell et al. (1993) J. Gen. Microbiol. 139(Pt. 1): 119-127; Lloret et al. (1996) Mol. Microbiol. 21: 449-457). By introducing a mutation that disrupts GDP-Man dehydrogenase activity, for example, one can obtain a cell that produces a higher level of GDP-Man than a wild-type cell. If a gene that encodes a glycosyltransferase that uses GDP-Man as a substrate is introduced into the cell, the GDP-Man that is no longer used for alginate synthesis is diverted to the synthesis of a desired mannosylated oligosaccharide. Alternatively, one can introduce genes that encode one or more enzymes such as those described above that convert GDP-Man to a different activated sugar, such as GDP-Fuc. The resulting recombinant cells can then be used for producing a fucosylated oligosaccharide of interest.

Similarly, one can construct a recombinant cell in which UDP-GlcNAc utilization is diverted from synthesis of peptidoglycan to synthesis of a desired GlcNAc-containing oligosaccharide. In E. coli, for example, a series of six enzymes, which act sequentially, are involved in conversion of UDP-GlcNAc into precursors of peptidoglycans (Mengin-Lecreulx et al. (1983) J. Bact. 154: 1284-1290). By disrupting one of these enzymes, preferably the first-acting enzyme, and introducing a GlcNAc transferase into the cell, one can divert the large quantities of UDP-GlcNAc produced by the cell to production of a desired GlcNAc-containing oligosaccharide. Alternatively, introduction of a gene encoding UDP-GlcNAc 4′-epimerase can result in conversion of UDP-GlcNAc to UDP-GalNAc, which can then serve as a sugar donor for a UDP-GalNAc transferase, which is encoded by an exogenous gene that is also introduced into the cell.

As another example, Escherichia sp., including E. coli, can produce a membrane-bound polysialic acid. Mutant strains in which synthesis of the polysialic acid is disrupted accumulate CMP-sialic acid (Vimr and Troy (1985) J. Bact. 164: 854-860; Gonzalez-Clemente et al. (1990) Biol. Chem. 371: 1101-1106; Cho et al. (1994) Proc. Nat'l. Acad. Sci. USA 91: 11427-11431). Introduction of a sialyltransferase gene into these mutant strains results in a recombinant cell that is capable of producing large amounts of a sialylated product saccharide. The extracellular polysaccharide colanic acid is also produced by E. coli, using GDP-fucose as a precursor. Accordingly, one can disrupt the activity of an enzyme involved in the conversion of GDP-fucose to colanic acid (e.g., GDP-Man 4,6-dehydratase; Stevenson et al. (1996) J. Bacteriol. 178: 4885-4893).

Bacteria belonging to the genera Azorhizobium, Bradyrhizobium, Rhizobium, and Sinorhizohium can produce lipo-chitooligosaccharides (LCOs). In at least some of these genera, a fucosyltransferase is encoded that uses GDP-fucose as a donor for transfer of fucose to LCO precursors (Mergaert et al. (1997) FEBS Lett. 409: 312-316). Accordingly, by disrupting the activity of this fucosyltransferase, one can divert the GDP-fucose produced by the cells to other uses. For example, a different fucosyltransferase gene can be introduced into the cells, thus obtaining a recombinant cell that produces a desired fucosylated saccharide.

Other examples of organisms and associated nucleotide sugars that one can divert to production of a desired saccharide by disruption of polymer synthesis are: Azotobacter vinelandii/GDP-Man; Pseudomonas sp./UDP-Glc and GDP-Man; Rhizobium sp./UDP-Glc, UDP-Gal, GDP-Man; Erwinia sp./UDP-Gal, UDP-Glc; Escherichia sp./UDP-GlcNAc, UDP-Gal, CMP-NeuAc, GDP-Fuc; Klebsiella sp./UDP-Gal, UDP-GlcNAc, UDP-Glc, UDP-GlcNAc (see, e.g., Hamadeh et al. (1996) Infect. Immun. 64: 528-534); Hansenula jadinii/GDP-Man, GDP-Fuc; Candida famata/UDP-Glc, UDP-Gal, UDP-GlcNAc (Ko et al. (1996) Appl. Biochem. Biotechnol. 60: 41-48); Acetobacter xylinum/GDP-Man (Petroni et al. (1996) J. Bacteriol. 178: 4814-4121) and Saccharomyces cerevisiae/UDP-Glc, UDP-Gal, GDP-Man, GDP-GlcNAc.

Methods of introducing mutations into a target gene are well known to those of skill in the art and are described in, for example, Ausubel, Sambrook, and Berger, all supra.

3. Fusion Proteins

In some embodiments, the recombinant cells of the invention express fusion proteins that have more than one enzymatic activity that is involved in synthesis of a desired oligosaccharide. The fusion polypeptides can be composed of, for example, a catalytic domain of a glycosyltransferase that is joined to a catalytic domain of an accessory enzyme. The accessory enzyme catalytic domain can, for example, catalyze a step in the formation of a nucleotide sugar which is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressable nucleotide sequence. The polypeptides of the present invention can be readily designed and manufactured utilizing various recombinant DNA techniques well known to those skilled in the art. Suitable fusion proteins are described in PCT Patent Application PCT/CA98/01180, which was published as WO99/31224 on Jun. 24, 1999.

4. Construction of Recombinant Cells.

The recombinant cells of the invention contain an exogenous gene that encodes a glycosyltransferase that catalyzes a desired glycosylation reaction, an enzymatic system for producing a nucleotide sugar that is a donor substrate for the glycosyltransferase, and an exogenous saccharide acceptor moiety. The glycosyltransferase catalyzes the transfer of a sugar from the nucleotide sugar to the acceptor moiety to produce the desired oligosaccharide.

In some embodiments, the enzymatic system for nucleotide sugar production also is modified by recombinant methods. For example, the enzymatic system can include one or more enzymes that are encoded by genes that are exogenous to the cell, or that are modified to increase the level of nucleotide sugar production, as discussed above.

Typically, the polynucleotide that encodes the exogenous glycosyltransferase or enzyme involved in nucleotide sugar synthesis is placed under the control of a promoter that is functional in the desired host cell. An extremely wide variety of promoters are well known, and can be used in the vectors of the invention, depending on the particular application. Ordinarily, the promoter selected depends upon the cell in which the promoter is to be active. Other expression control sequences such as ribosome binding sites, transcription termination sites and the like are also optionally included. Expression control sequences that are suitable for use in a particular host cell are often obtained by cloning a gene that is expressed in that cell. The recombinant cells of the invention can be plant cells or microorganisms, such as, for example, yeast cells, bacterial cells, or fungal cells. Examples of suitable cells include, for example, Azotobacter sp. (e.g., A. vinelandii), Pseudomonas sp., Rhizobium sp., Erwinia sp., Escherichia sp. (e.g., E. coli), and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g, H. anomala and H jadinii), Brettanomyces (e.g., B. lambicus and B. anomalus), and tobacco.

A promoter and other control signals can be derived from a gene that is under investigation, or can be a heterologous promoter or other signal that is obtained from a different gene, or from a different species. Where continuous expression of a gene is desired, one can use a “constitutive” promoter, which is generally active under most environmental conditions and states of development or cell differentiation. Suitable constitutive promoters for use in plants include, for example, the cauliflower mosaic virus (CaMV) 35S transcription initiation region and region VI promoters, the 1′- or 2′- promoter derived from T-DNA of Agrobacterium tumefaciens, and other promoters active in plant cells that are known to those of skill in the art. Other suitable promoters include the full-length transcript promoter from Figwort mosaic virus, actin promoters, histone promoters, tubulin promoters, or the mannopine synthase promoter (MAS). Other constitutive plant promoters include various ubiquitin or polyubiquitin promoters derived from, inter alia, Arabidopsis (Sun and Callis, Plant J., 11(5):1017-1027 (1997)), the mas, Mac or DoubleMac promoters (described in U.S. Pat. No. 5,106,739 and by Comai et al., Plant Mol. Biol. 15:373-381 (1990)) and other transcription initiation regions from various plant genes known to those of skill in the art. Such genes include for example, ACT11 from Arabidopsis (Huang et al., Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al., Plant Physiol. 104:1167-1176 (1994)), GPc1 from maize (GenBank No. X1 5596, Martinez et al., J. Mol. Biol 208:551-565 (1989)), and Gpc2 from maize (GenBank No. U45855, Manjunath et al., Plant Mol. Biol. 33:97-112 (1997)). Useful promoters for plants also include those obtained from Ti- or Ri-plasmids, from plant cells, plant viruses or other hosts where the promoters are found to be functional in plants. Bacterial promoters that function in plants, and thus are suitable for use in the methods of the invention include the octopine synthetase promoter, the nopaline synthase promoter, and the manopine synthetase promoter. Suitable endogenous plant promoters include the ribulose-1,6-biphosphate (RUBP) carboxylase small subunit (ssu) promoter, the ((x-conglycinin promoter, the phaseolin promoter, the ADH promoter, and heat-shock promoters.

Promoters for use in E. coli include the T7, trp, or lambda promoters. A ribosome binding site and preferably a transcription termination signal are also provided. For eukaryotic cells, the control sequences typically include a promoter which optionally includes an enhancer derived from immunoglobulin genes, SV40, cytomegalovirus, etc., and a polyadenylation sequence, and may include splice donor and acceptor sequences.

In yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). For filamentous fungi such as, for example, strains of the fungi Aspergillus (McKnight et al., U.S. Pat. No. 4,935,349), examples of useful promoters include those derived from Aspergillus nidulans glycolytic genes, such as the ADH3 promoter (McKnight et al., EMBO J. 4: 2093 2099 (1985)) and the tplA promoter. An example of a suitable terminator is the ADH3 terminator (McKnight et al.).

In some embodiments, the polynucleotides are placed under the control of an inducible promoter, which is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the glycosyltransferase or enzyme involved in nucleotide sugar synthesis. For E. coli and other bacterial host cells, inducible promoters are known to those of skill in the art. These include, for example, the lac promoter. A particularly preferred inducible promoter for expression in prokaryotes is a dual promoter that includes a tac promoter component linked to a promoter component obtained from a gene or genes that encode enzymes involved in galactose metabolism (e.g., a promoter from a UDPgalactose 4-epimerase gene (galE)). The dual tac-gal promoter, which is described in U.S. Ser. No. 08/965,850, filed Nov. 7, 1997, provides a level of expression that is greater than that provided by either promoter alone.

Inducible promoters for use in plants are known to those of skill in the art (see, e.g., references cited in Kuhlemeier et al (1987) Ann. Rev. Plant Physiol. 38:221), and include those of the 1,5-ribulose bisphosphate carboxylase small subunit genes of Arabidopsis thaliana (the “ssu” promoter), which are light-inducible and active only in photosynthetic tissue, anther-specific promoters (EP 344029), and seed-specific promoters of, for example, Arabidopsis thaliana (Krebbers et al. (1988) Plant Physiol. 87:859).

Inducible promoters for other organisms are also well known to those of skill in the art. These include, for example, the arabinose promoter, the lacZ promoter, the metallothionein promoter, and the heat shock promoter, as well as many others.

A construct that includes a polynucleotide of interest operably linked to gene expression control signals that, when placed in an appropriate host cell, drive expression of the polynucleotide is termed an “expression cassette.” Expression cassettes that encode the glycosyltransferase and/or enzyme involved in nucleotide sugar synthesis are often placed in expression vectors for introduction into the host cell. The vectors typically include, in addition to an expression cassette a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in U.S. Ser. No. 08/965.850, filed Nov. 7, 1997.

The construction of polynucleotide constructs generally requires the use of vectors able to replicate in bacteria. A plethora of kits are commercially available for the purification of plasmids from bacteria. For their proper use, follow the manufacturer's instructions (see, for example, EasyPrepJ, FlexiPrepJ, both from Pharmnacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to construct the cells of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the target cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in culture medium, thus removing selective pressure and allowing the culture to become overgrown with cells that do not contain the vector.

Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l. Acad. Sci. USA 78: 2072; Southern & Berg (1982) J. Mol. Appl. Genet. 1: 327).

Selection markers for plant and/or other eukaryotic cells often confer resistance to a biocide or an antibiotic, such as, for example, kanamycin, G 418, bleomycin, hygromycin, or chloramphenicol, or herbicide resistance, such as resistance to chlorsulfuron or Basta. Examples of suitable coding sequences for selectable markers are: the neo gene which codes for the enzyme neomycin phosphotransferase which confers resistance to the antibiotic kanamycin (Beck et al (1982) Gene 19:327); the hyg gene, which codes for the enzyme hygromycin phosphotransferase and confers resistance to the antibiotic hygromycin (Gritz and Davies (1983) Gene 25:179); and the bar gene (EP 242236) that codes for phosphinothricin acetyl transferase which confers resistance to the herbicidal compounds phosphinothricin and bialaphos.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable for constructing the recombinant cells of the invention are well known in the art. For cloning in bacteria, common vectors include pBR322 derived vectors such as pBLUESCRIPT™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses).

The methods for introducing the expression vectors into a chosen host cell are not particularly critical, and such methods are known to those of skill in the art. For example, the expression vectors can be introduced into prokaryotic cells, including E coli, by calcium chloride transformation, and into eukaryotic cells by calcium phosphate treatment or electroporation. Other transformation methods are also suitable.

B. Reaction Mixtures and Methods for Synthesizing Product Saccharides

The invention also provides reaction mixtures and methods in which the recombinant cells of the invention are used to prepare product saccharides (which are composed of two or more saccharide residues). The recombinant cells used in the reaction mixtures express at least one glycosyltransferase and a nucleotide sugar that functions as a sugar donor for the glycosyltransferase. The reaction mixtures also include an acceptor saccharide to which the glycosyltransferase can transfer the sugar to form a desired oligosaccharide.

The recombinant cells of the invention are grown in culture to obtain a sufficient number of cells for use in a reaction of a desired scale. Methods and culture media for growth of the respective host cells are well known to those of skill in the art. Culture can be conducted in, for example, aerated spinner or shaking culture, or, more preferably, in a fermentor.

Upon growth of the recombinant cells to a desired cell density, the cells are typically processed for use in the reaction mixtures and methods of the invention. For example, the cells are generally permeabilized or otherwise disrupted to allow entry of the saccharide acceptors into the cells. The glycosyltransferase and nucleotide sugar produced by the cells can, in some situations, diffuse from the cells into the extracellular fluid. Methods of permeabilizing cells so as to not significantly degrade enzymatic activity and nucleotide sugar stability are known to those of skill in the art. Cells can be subjected to concentration, drying, lyophilization, treatment with surfactants, ultrasonic treatment, mechanical disruption, enzymatic treatment, and the like.

The treated cells are then used in a reaction mixture that contains additional reactants, known to those of skill in the art, that are necessary or desirable for the enzymatic activity of the glycosyltransferase. The concentration of treated cells used in the reaction mixture is typically between about 0.1% (wet wt/vol) and 50% (wet wt/vol), more preferably between about 1% (wet wt/vol) and about 20% (wet wt/vol), and most preferably between about 2% (wet wt/vol) and about 10% (wet wt/vol), or a corresponding amount of dry cells.

The reaction mixtures also include a saccharide acceptor. Suitable acceptors for sialyltransferases, for example, generally include a Gal residue, and include, for example, Galβ1→3GalNAc, lacto-N-tetraose, Galβ1→3GlcNAc, Galβ1→3Ara, Galβ1→6GlcNAc, Galβ1→4Glc (lactose), Galβ1→4Glcβ1-OCH₂CH₃, Galβ1→4Glcβ1-OCH₂CH₂CH₃, Galβ1→4Glcβ1-OCH₂C₆H₅, Galβ1→4GlcNAc, Galβ1-OCH₃, melibiose, raffinose, stachyose, and lacto-N-neotetraose (LNnT). Sialyltransferases that are used in the recombinant cells and reaction mixtures of the invention are, in some embodiments, able to transfer sialic acid to the sequence Galβ1,4GlcNAc-, the most common penultimate sequence underlying the terminal sialic acid on fully sialylated carbohydrate structures. Only three of the cloned mammalian sialyltransferases meet this acceptor specificity requirement, and each of these have been demonstrated to transfer sialic acid to N-linked carbohydrate groups of glycoproteins. Examples of sialyltransferases that use Galβ1,4GlcNAc as an acceptor are shown in Table 1.

TABLE 1
Sialyltransferases which use the Galβ1,4GlcNAc saccharide as
an acceptor substrate.

| Sialyltransferase | Source | Structure formed | Ref. |
|---|---|---|---|
| ST6Gal I | Mammalian | NeuAcα2,6Galβ1,4GlcNAc- | 1 |
| ST3Gal III | Mammalian | NeuAcα2,3Galβ1,4GlcNAc- | 1 |
| | | NeuAcα2,3Galβ1,3GlcNAc- | |
| ST3 Gal IV | Mammalian | NeuAcα2,3Galβ1,4GlcNAc- | 1 |
| | | NeuAcα2,3Galβ1,3GlcNAc- | |
| ST6Gal II | Photobacterium | NeuAcα2,6Galβ1,4GlcNAc- | 2 |
| ST3Gal V | N. meningitides | NeuAcα2,3Galβ1,4GlcNAc- | 3 |
| | N. gonorrhoeae | | |

1 Goochee et al. (1991) Bio/Technology 9: 1347-1355
2 Yamamoto et al. (1996) J. Biochem. 120: 104-110
3 Gilbert et al. (1996) J. Biol. Chem. 271: 28271-28276

For sialyltransferase nomenclature, see Tsuji et al. (1996) Glycobiology 6: v-xiv).

Other ingredients can include a divalent cation (e.g, Mg⁺² or Mn⁺²), materials necessary for ATP regeneration, phosphate ions, and organic solvents. The concentrations or amounts of the various reactants used in the processes depend upon numerous factors including reaction conditions such as temperature and pH value, and the choice and amount of acceptor saccharides to be glycosylated. The reaction medium can also contain solubilizing detergents (e.g., Triton or SDS) and organic solvents such as methanol or ethanol, if necessary.

The temperature at which an above process is carried out can range from just above freezing to the temperature at which the most sensitive enzyme denatures. That temperature range is preferably about zero degrees C. to about 110° C., and more preferably at about 20° C. to about 30° C., or higher for a thermophilic organism.

The reaction mixture so formed is maintained for a period of time sufficient for the donor saccharide to be added to the acceptor. Some of the product can often be detected after a few hours, with recoverable amounts usually being obtained within 24 hours. It is preferred to optimize the yield of the process, and the maintenance time is usually about 36 to about 240 hours.

The products produced by the above processes can be used without purification. However, it is usually preferred to recover the product. Standard, well known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins. Nanofiltration or reverse osmosis can then be used to remove salts and/or purify the product saccharides (see, e.g., U.S. patent application No. 08/947,775, filed Oct. 9, 1997). Nanofilter membranes are a class of reverse osmosis membranes which pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

The methods of the invention are capable of producing large amounts of a desired product saccharide. For example, one can produce a product saccharide to a final concentration of about 1 mM or greater. More preferably, the product saccharide is produced at a concentration of about 2.5 mM or greater, still more preferably at about 5 mM or greater, and most preferably the reaction methods of the invention produce the product saccharide at a concentration of about 10 mM or greater.

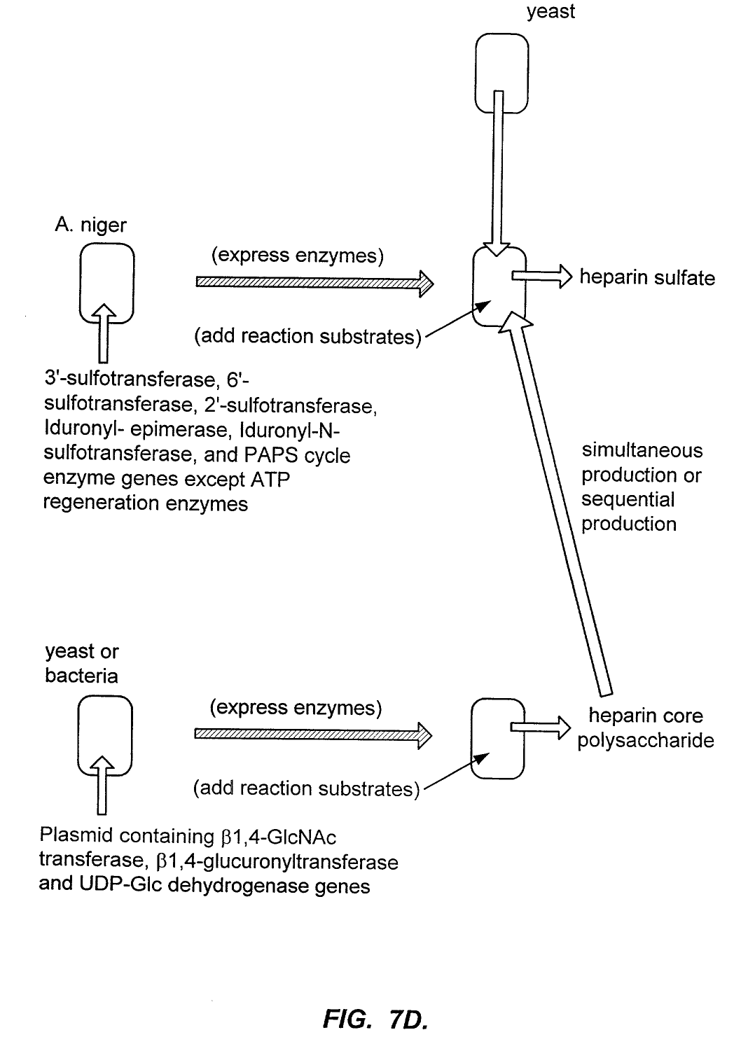

This approach can be used to produce the active sulfating agent PAPS for producing sulfated sugars (see, e.g., FIGS. 7A-D). Incorporation of genes that encode a sulfotransferase or multiple sulfotransferases into an organism that produces PAPS, either naturally or through the addition of the PAPS cycle regeneration enzymes, will allow the sulfation of oligosaccharides or polysaccharides. This process can be performed either by addition of the sugar to be sulfated to this PAPS sulfating organism or by addition of the PAPS containing organism to other organisms that are capable of forming the glycosidic linkages of the sugar of interest. PAPS enzymes can be introduced either by genomic insertion into the organism or via plasmids capable of producing the enzyme activity of interest. As an example, if the PAPS cycle enzymes and three sulfotransferases required for heparan or heparin sulfation are added to an organism and either the backbone unsulfated polysaccharide of heparan or heparin is added to this organism under appropriate conditions, then the polysaccharide will be sulfated producing sulfated heparan or heparin.

In some embodiments, the reaction mixture includes two or more types of recombinant cells. For example, an organism that produces a nucleotide triphosphate necessary for a cycle reaction can be combined with an organism that contains all of the remaining cycle enzymes necessary to produce the glycosidic linkage of interest (see, e.g., FIGS. 5A and 5B). Once combined, the two organisms work together to complete the cycle and produce the nucleotide sugar of interest. An illustrative example involves the combination of a bacteria such as Corynebacterium, which produces UTP, with an E. coli strain that contains one or more plasmids that encode the remaining enzymes of the GlcNAc cycle (Table 1). In FIG. 5A, the Corynebacterium strain naturally produces UTP from UDP; after the glycosyltransferase reaction, the UDP that is released by the reaction in the E. coli diffuses back into the Corynebacterium, where UTP is regenerated. The two organisms are permeabilized and the starting reagents of, for example, glucose, orotic acid, GlcNAc and lactose are added; the end product in this example is LNT-2. In FIG. 5B, the Corynebacterium does not produce sufficient CTP, so a CTP-synthetase gene is introduced into the cell which catalyzes the formation of CTP. The CTP diffuses into the E. coli cell, which contains an exogenous gene that encodes a fusion protein in which the catalytic domain of a 3′-sialyltransferase is linked to the catalytic domain for CMP-sialic acid synthetase. Also present in the E. coli cells are genes that encode GlcNAc epimerase and NeuAc aldolase. Yeast (for example, bakers yeast) can also be used to regenerate CTP from CMP using glucose, phosphate and CMP as the reagents.

In another approach, each of the two or more cell types used in a reaction mixture produces a different glycosyltransferase and corresponding nucleotide sugar. Combinations of the recombinant cells of the invention, each producing a nucleotide sugar or multiple nucleotide sugars and one or more glycosidic linkages, can be combined either sequentially or simultaneously to produce a sugar containing new multiple glycosidic linkages. Thus, the invention provides a simple method for producing oligosaccharides with multiple linkages or polysaccharides and related polymeric structures.

The generation and possible regeneration of the sugar nucleotide, nucleotide or PAPS in the organism, either produced via natural pathways or from incorporated cycle enzymes, can be energized using the organism's natural metabolic pathways to produce high energy intermediates such as PEP, acetylphosphate, ATP, creatinephosphate, etc., or by adding additional enzymes capable of producing similar intermediates. The energy for regeneration is therefore provided by such molecules as simple sugars (e.g., glucose, fructose, maltose, sucrose, etc), polyphosphate, pyruvate, alcohols, fats or fatty acids, amino acids, and the like. Glycosyltransferase cycles are described in, for example, U.S. Pat. Nos. 5,876,980, 5,728,554, and 5,922,577, as well as PCT Patent No. 96/04790.

C. Uses for the Recombinant Cells and Reaction Mixtures

The recombinant cells, reaction mixtures, and methods of the invention are useful for synthesizing a wide range of product saccharides that have many uses. Products that can be produced using this method include, for example, disaccharides, oligosaccharides, polysaccharides, lipopolysaccharides, glycoproteins, glycopeptides, and glycolipids including gangliosides. Any glycosidic linkage can be made using this approach. Such linkages include, but are not limited to, the addition of such sugars as fucose, sialic acid, galactose, GlcNAc, GalNAc, mannose, glucose, uronic acid forms of these sugars (e.g., glucuronic acid, galacturonic acid, etc.), xylose and fructose.

Product saccharides that can be produced using the methods and reaction mixtures of the invention and are of particular interest include, but are not limited to:

1. Oligosaccharides

The reaction mixtures and methods are useful for producing a wide range of oligosaccharides, including sialyllactose, fucosyllactose, GalNAc-lactose, GlcNAclactose, LNnT, LNT, LNT-2, fucosyl-LNnT, fucosyl-LNT, sialyl-LNnT (LSTd), sialyl-LNT, GalNAc-LNnT, α1,3-Gal-Lactose, α1,3-Gal-N-acetyllactosamine, STn-antigen, Tn-antigen, T-antigen. heparans, and glycosides thereof. The glycosides can include incorporation of linker arms or the like for coupling to other materials.

In some embodiments, the recombinant cells and reaction mixtures are constructed for production of a fucosylated saccharide product. Through use of a cell that produces GDP-fucose and contains the appropriate fucosyltransferase enzymes, the following carbohydrate structures are among those that one can obtain: (1) Fucα(1→2) Galβ-; (2) Galβ(1→3)[Fucα(1→4)]GlcNAcβ-; (3) Galβ(1→4)[Fucα(1→3)]GlcNAcβ-; (4) Galβ(1→4)[Fucα(1→3)]Glc; (5)-GlcNAcβ(1→4)[Fucα(1→6)]GlcNAcβ1-Asn; (6)-GlcNAcβ(1→4)[Fucα(1→3)GlcNAcβ1→Asn; (7) Fucα(1→6)Galβ→; (8) Fucα(1→3) Galβ-; (9) Glcβ(1→3)Fucα1→O-Thr and Fucα1→O-Thr/Scr; and (10) Fucα1→Ceramide. Examples of products that can be formed using GDP-fucose as a reactant include, but are not limited to, those listed in Table 2.

TABLE 2
Oligosaccharide Structures Synthesized using GDP-
fucose and Fucosyltransferase

| Oligosaccharide | Tissue source |
|---|---|
| III³ Fucosyl-para-lacto-N-hexaose | Human milk |
| 3′-Sialyl-3-fucosyllactose | Human milk |
| Lewis X | hematopoietic cells |
| Lewis A | hematopoietic cells |
| Sialyl lewis X | hematopoietic cells |
| Sialyl lewis A | hematopoietic cells |
| Lacto-N-difucohexaose II | Human milk |
| Lacto-N-fucopentaose I | Human milk |
| Lacto-N-fucopentaose II | Human milk |
| 2′-Fucosyllactose | Human milk |
| Lactodifucotetraose | Human milk |
| 3-Fucosyllactose | Human milk |
| Lacto-N-fucopentaose III | Human milk |
| Lacto-N-difucohexaose I | Human milk |
| Lacto-N-fucapentaose V | Human milk |

Galactosides can also be produced using the recombinant cells and methods of the invention. For example, by use of a recombinant cell that produces UDP-Gal and contains the appropriate galactosyltransferase, one can add Gal in a β1,4 linkage, an α1,3 linkage, an α1,4 linkage, or a β1,3 linkage to a saccharide that includes a GlcNAc or Glc residue. The recombinant cells are permeabilized and placed in contact with the acceptor saccharide, resulting of transfer of the Gal from the UDP-Gal to the acceptor. One example of such an oligosaccharide for which the invention provides an efficient method of synthesis is lacto-N-neotetraose, Galβ(1-4)-GlcNAcβ(1-3)-Galβ(1-4)-Glc (formula I). See, e.g. Min-Yuan Chou et al. (1996) J. Biol. Chem. 271 (32): 19166-19173.

[chemical structure]

The invention also provides methods for adding GalNAc or GlcNAc to Gal, in a β1,3 linkage or a β1,4 linkage, by providing a recombinant cell that encodes a GalNAc transferase or GlcNAc transferase and which produces an activated UDP-GalNAc or UDP-GlcNAc. The cells are disrupted and placed in contact with an acceptor moiety that includes a Gal residue.

The recombinant cells and reaction mixtures of the invention are particularly useful in synthesizing product saccharides that require multiple enzymatic steps. In these embodiments, the a recombinant cell can contain two or more exogenous glycosyltransferase genes, and produce both of the respective nucleotide sugar substrates. Alternatively, a reaction mixture can contain two or more types of recombinant cells, each of which contains one or more exogenous glycosyltransferase genes and the corresponding nucleotide sugar generating system. For example, one can use a combination of recombinant cell types, one of which contains an exogenous sialyltransferase gene and a system for producing CMP-sialic acid, and another recombinant cell type that contains an exogenous galactosyltransferase gene and produces UDP-Gal. In this group of embodiments, the different cell types can be combined in an initial reaction mixture, or preferably the recombinant cell types for a second glycosyltransferase reaction can be added to the reaction medium once the first glycosyltransferase reaction has neared completion. By conducting two glycosyltransferase reactions in sequence in a single vessel, overall yields are improved over procedures in which an intermediate species is isolated. Moreover, cleanup and disposal of extra solvents and by-products is reduced.

For example, the present invention provides recombinant cells and methods for the preparation of compounds having the formula:

NeuAcα(2→3)Galβ(1→4)(Fucα1→3)GlcN(R′)β(1→3)Galβ-OR

In this formula, R is a hydrogen, a saccharide, an oligosaccharide or an aglycon group having at least one carbon atom. R′ can be either acetyl or allyloxycarbonyl (Alloc).

The term “aglycon group having at least one carbon atom” refers to a group -A-Z, in which A represents an alkylene group of from 1 to 18 carbon atoms optionally substituted with halogen, thiol, hydroxy, oxygen, sulfur, amino, imino, or alkoxy; and Z is hydrogen, —OH, —SH, —NH₂, —NHR¹, —N(R¹)₂, —CO₂H, —CO₂R¹, —CONH₂, —CONHR¹, —CON(R¹)₂, —CONHNH₂, or —OR¹ wherein each R¹ is independently alkyl of from 1 to 5 carbon atoms. In addition, R can be

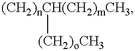

where n,m,o=1-18: (CH₂)ₙ-R² (in which n=0-18), wherein R² is a variously substituted aromatic ring, preferably, a phenyl group, being substituted with one or more alkoxy groups, preferably methoxy or O(CH₂)ₘCH₃, (in which m=0-18), or a combination thereof.

The steps involved in synthesizing these compounds include:

(a) galactosylating a compound of the formula GlcNR′β(1→3)Galβ-OR with a galactosyltransferase in the presence of a UDP-galactose under conditions sufficient to form the compound: Galβ(1→4)GlcNR′β(1→3)Galβ-OR;

(b) sialylating the compound formed in (a) with a sialyltransferase in the presence of a CMP derivative of a sialic acid using a α(2,3)sialyltransferase under conditions in which sialic acid is transferred to the non-reducing sugar to form the compound: NeuAcα(2→3)Galβ(1→4)GlcNR′β(1→3)Galβ-OR; and

(c) fucosylating the compound formed in (b) to provide the NeuAcα(2→3)Galβ(1→4)(Fucα1→3)GlcNR′β(1→3)Galβ-OR.

The recombinant cells of the invention provide an efficient way to carry out each of these steps, either individually or simultaneously. One or more of the steps can be conducted using the recombinant cells of the invention. For example, the galactosylation reaction can be accomplished using a recombinant cell that contains an exogenous galactosyltransferase gene and which produces UDP-Gal. The sialylation and fucosylating steps can also be carried out using recombinant cells that produce the appropriate glycosyltransferase and donor sugar, or can be carried out using conventional non-cell-based methods. In a presently preferred embodiment, at least two of the reaction steps are carried out using recombinant cells of the invention. The different glycosyltransferases and respective nucleotide sugar synthesizing systems can be present in the same cell, or different recombinant cells which each contain an exogenous glycosyltransferase gene and respective nucleotide sugar generating system can be mixed together. Thus, by mixing and matching members of a set of recombinant cells, each of which contain a different glycosyltransferase and corresponding nucleotide sugar generating system, one can readily create a custom reaction mixture for performing many multi-step glycosylation reactions.

In a particularly preferred embodiment, R is ethyl, the fucosylation step is carried out chemically, and the galactosylation and sialylation steps are carried out in a single vessel.

Among the compounds that one can produce using the recombinant cells, reaction mixtures, and methods of the invention are sialic acid and any sugar having a sialic acid moiety. These include the sialyl galactosides, including the sialyl lactosides, as well as compounds having the formula:

NeuAcα(2→3)Galβ(1→4)GlcN(R′)β-OR or

NeuAcα(2→3)Galβ(1→4)GlcN(R′)β(1→3)Galβ-OR

In these formulae, R′ is alkyl or acyl from 1-18 carbons, 5,6,7,8-tetrahydro-2-naphthamido; benzamido; 2-naphthamido; 4-aminobenzamido; or 4-nitrobenzamido. R is a hydrogen, a alkyl C₁-C₆, a saccharide, an oligosaccharide or an aglycon group having at least one carbon atom. The term “aglycon group having at least one carbon atom” refers to a group -A-Z, in which A represents an alkylene group of from 1 to 18 carbon atoms optionally substituted with halogen, thiol, hydroxy, oxygen, sulfur, amino, imino, or alkoxy; and Z is hydrogen, —OH, —SH, —NH₂, —NHR¹, —N(R¹)₂, CO₂H, CO₂R¹, —CONH₂, —CONHR¹, —CON(R¹)₂, —CONHNH₂, or —OR¹ wherein each R¹ is independently alkyl of from 1 to 5 carbon atoms. In addition, R can be

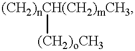

where n,m.o=1-18; (CH₂)ₙ-R² (in which n=0-18), wherein R² is a variously substituted aromatic ring, preferably, a phenyl group, being substituted with one or more alkoxy groups, preferably methoxy or O(CH₂)ₘCH₃, (in which m=0-18), or a combination thereof. R can also be 3-(3,4,5-trimethoxyphenyl)propyl.

A related set of structures included in the general formula are those in which Gal is linked β1,3 and Fuc is linked α1,4. For instance, the tetrasaccharide, NeuAcα2,3Galβ1,3(Fucα1,4)GlcNAcβ1-, termed here SLe^{a}, is recognized by selecting receptors. See, Berg et al., J. Biol. Chem., 266:14869-14872 (1991). In particular, Berg et al. showed that cells transformed with E-selecting cDNA selectively bound neoglycoproteins comprising SLe^{a}.

The methods of the invention are also useful for synthesizing oligosaccharide compounds having the general formula Galα1,3Gal-, including Galα1,3Galβ1,4Glc(R)β-O-R¹, wherein R¹ is —(CH₂)ₙ—COX, with X═OH, OR², —NHNH₂, R═OH or NAc, and R² is a hydrogen, a saccharide, an oligosaccharide or an aglycon group having at least one carbon atom, and n=an integer from 2 to 18, more preferably from 2 to 10. Also among the compounds that can be synthesized according to the invention are lacto-N-neotetraose (LNnT), GlcNAcβ1,3Galβ1,4Glc (LNT-2), sialyl(α2,3)-lactose, and sialyl(α2,6)-lactose.

In the above descriptions, the terms are generally used according to their standard meanings. The term “alkyl” as used herein means a branched or unbranched, saturated or unsaturated, monovalent or divalent, hydrocarbon radical having from 1 to 20 carbons, including lower alkyls of 1-8 carbons such as methyl, ethyl, n-propyl, butyl, n-hexyl, and the like, cycloalkyls (3-7 carbons), cycloalkylmethyls (4-8 carbons), and arylalkyls. The term “alkoxy” refers to alkyl radicals attached to the remainder of the molecule by an oxygen, e.g., ethoxy, methoxy, or n-propoxy. The term “alkylthio” refers to alkyl radicals attached to the remainder of the molecule by a sulfur. The term of “acyl” refers to a radical derived from an organic acid by the removal of the hydroxyl group. Examples include acetyl, propionyl, oleoyl, myristoyl.

The term “aryl” refers to a radical derived from an aromatic hydrocarbon by the removal of one atom, e.g., phenyl from benzene. The aromatic hydrocarbon may have more than one unsaturated carbon ring, e.g., naphthyl.

The term “alkoxy” refers to alkyl radicals attached to the remainder of the molecule by an oxygen, e.g., ethoxy, methoxy, or n-propoxy.

The term “alkylthio” refers to alkyl radicals attached to the remainder of the molecule by a sulfur.

An “alkanoamido” radical has the general formula —NH—CO—(C₁-C₆ alkyl) and may or may not be substituted. If substituted, the substituent is typically hydroxyl. The term specifically includes two preferred structures, acetamido, —NH—CO—CH₃, and hydroxy acetamido, —NH—CO—CH₂—OH.

The term “heterocyclic compounds” refers to ring compounds having three or more atoms in which at least one of the atoms is other than carbon (e.g., N, O, S, Se, P, or As). Examples of such compounds include furans (including the furanose form of pentoses, such as fucose), pyrans (including the pyranose form of hexoses, such as glucose and galactose) primidines, purines, pyrazines and the like.

2. Glycolipids, Including Gangliosides and Related Structures

The reaction mixtures and cells of the invention are also useful for producing many different glycolipids. Those of particular interest include, for example, Lactosylceramide, glucosylceramide, Globo-H, Globotetrose, lipopolysaccharides and various forms of these lipids. For example, the lipids can be modified to be, for example, a lyso-, deacetyl, linker arm-containing, or an O-acetyl forms.

The invention provides reaction mixtures, cell types, and methods for adding one or more saccharide moieties in a specific manner in order to obtain a desired ganglioside or other glycosphingolipid, or derivatives thereof. The methods of the invention involve the use of cells that express one or more recombinant glycosyltransferases to synthesize glycosphingoids, including gangliosides and other glycosphingoids. Through use of a glycosyltransferase to link a desired carbohydrate to the precursor molecule, one can achieve a desired linkage with high specificity. In some embodiments, it is desirable to remove the fatty acid moiety from the sphingoid precursor prior to the glycosyltransferase reaction, and/or to use an organic solvent to facilitate the reaction. Enzymes and reaction schemes for producing many gangliosides and related structures are described in co-pending, commonly assigned PCT Patent Application No. PCT/US/25470, which was published on Jun. 10, 1999 as Publication No. WO99/28491 and is entitled “Enzymatic synthesis of gangliosides.”

The methods of the invention are useful for producing any of a large number of gangliosides and related structures. Many gangliosides of interest are described in Oettgen, H. F., ed., Gangliosides and Cancer, VCH, Germany, 1989, pp. 10-15, and references cited therein. Gangliosides of particular interest include, for example, those found in the brain as well as other sources which are listed in Table 3.

TABLE 3
Ganglioside Formulas and Abbreviations

| Structure | Abbreviation |
|---|---|
| Neu5Ac3Gal4GlcCer | GM3 |
| GalNAc4(Neu5Ac3)Gal4GlcCer | GM2 |
| Gal3GalNAc4(Neu5Ac3)Gal4GlcCer | GM1a |
| Neu5Ac3Gal3GalNAc4Gal4GlcCer | GM1b |
| Neu5Ac8Neu5Ac3Gal4GlcCer | GD3 |
| GalNAc4(Neu5Ac8Neu5Ac3)Gal4GlcCer | GD2 |
| Neu5Ac3Gal3GalNAc4(Neu5Ac3)Gal4GlcCer | GD1a |
| Neu5Ac3Gal3(Neu5Ac6)GalNAc4Gal4GlcCer | GD1α |
| Gal3GalNAc4(Neu5Ac8Neu5Ac3)Gal4GlcCer | GD1b |
| Neu5Ac8Neu5Ac3Gal3GalNAc4(Neu5Ac3)Gal4GlcCer | GT1a |
| Neu5Ac3Gal3GalNAc4(Neu5Ac8Neu5Ac3)Gal4GlcCer | GT1b |

| | Abbre- |
|---|---|
| Structure | viation |
| Gal3GalNAc4(Neu5Ac8Neu5Ac8Neu5Ac3)Gal4GlcCer | GT1c |
| Neu5Ac8Neu5Ac3Gal3GalNAc4(Neu5Ac8Neu5c3)Gal4GlcCer | GQ1b |

Nomenclature of Glycolipids, IUPAC-IUB Joint Commission on Biochemical Nomenclature (Recommendations 1997); Pure Appl. Chem. (1997) 69: 2475-2487; Eur. J. Biochem (1998) 257: 293-298) (www.chem.qmw.ac.uk/iupac/misc/glylp.html).

3. Glycopeptides

In some embodiments, the product saccharides are attached to polypeptides. The reaction mixtures and cells of the invention are thus useful for modifying glycoproteins to achieve various improvements in properties such as therapeutic half-life, immunogenicity, and the like. Examples of glycopeptides of particular interest include, for example, STn-peptide, Tn-peptide, T-peptide, ST-peptide, and the linked versions of these structures. Enzymes and reactions that are useful for modification of glycoproteins are described in, for example, PCT Patent Application No. US98/00835, which was published as WO98/31826 on Jul. 23, 1998.

4. Polysaccharides

Product saccharides that can be synthesized using the reaction mixtures and cells of the invention include, for example, heparins, heparans, chondroitins, hyaluronic acid, dermatans, keratans, carragenans, alginates, agars, guar gums, fructans, glucans, cellulose, chitin, and chitosan. The desulfated, acetylated, anhydro or derivatized forms of each of these products can also be synthesized.

In some embodiments, the recombinant cells and reaction mixtures are used to synthesize sulfated polysaccharides, including heparin sulfate, heparan sulfate, and carragenan sulfate. Many biological processes involve sulfated biomolecules (U.S. Pat. No. 5,919,673; Varki (1993) Glycobiology3: 97). For example, sialyl Lewis X (SLe^{x}) which has a sulfate group at the 6-position of galactose is a ligand for L-selecting Hemmerich et al. (1994) Biochemisty 33: 4830), and sulfated Lewis a (Le^{a}) tetra and pentasaccharides are potent inhibitors of E-selecting binding (Yuen et al. (1994) J. Biol. Chem. 269: 1595). Other sulfated molecules that are involved in numerous cellular functions are the glycosaminoglycans (van Boeckel et al. (1993) Angew. Chem. Int. Ed. Eng. 32:1671. Hydroxysteroid sulfation provides hydrophilic forms for excretion) Ogura et al. (1989) Biochem. Biophys. Res. Commun. 165:169. Heparan sulfate proteoglycans on the cell surface bind and modulate biological activities of various growth factors, enzymes and protease inhibitors.

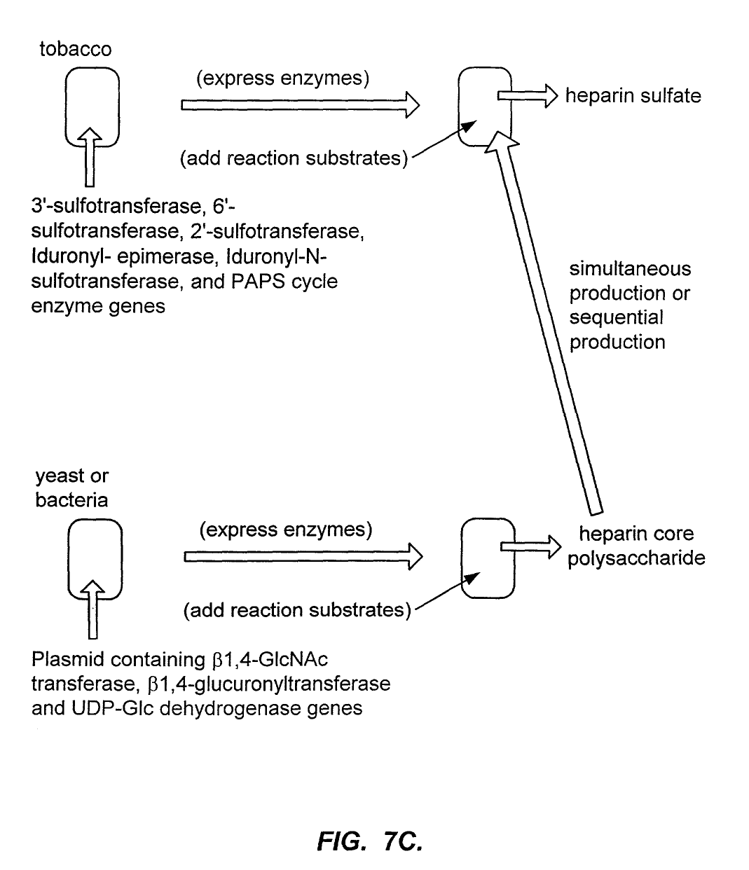

Preferably, these reactions employ a cell type that can produce the sulfate donor PAPS (3′-phosphoadenosine-5′-phosphosulfate). PAPS can serve as a sulfate donor for sulfotransferases, which can catalyze the sulfation of oligosaccharides and steroids. U.S. Pat. No. 5,919,673 describes a PAPS regeneration cycle that involves the use of several enzymes (FIG. 6). Examples of reaction schemes of the invention in which the PAPS regeneration cycle is used are shown in FIG. 7A-D. Enzymes that are involved in biosynthesis of heparin are described in, for example, Salmivirta et al. (1996) FASEB J. 10: 1270-1279.

5. Pharmaceutical and Other Applications

The compounds described above can then be used in a variety of applications, e.g., as antigens, diagnostic reagents, foodstuffs, or as therapeutics. Thus, the present invention also provides pharmaceutical compositions which can be used in treating a variety of conditions. The pharmaceutical compositions are comprised of oligosaccharides made according to the methods described above.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions are intended for parenteral, intranasal, topical, oral or local administration, such as by aerosol or transdermally, for prophylactic and/or therapeutic treatment. Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the oligosaccharides of the invention can be incorporated into liposomes formed from standard vesicle-forming lipids. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

The compositions containing the oligosaccharides can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as a “therapeutically effective dose.” Amounts effective for this use will depend on the severity of the disease and the weight and general state of the patient, but generally range from about 0.5 mg to about 40 g of oligosaccharide per day for a 70 kg patient, with dosages of from about 5 mg to about 20 g of the compounds per day being more commonly used.

Single or multiple administrations of the compositions can be carried out with dose levels and pattern being selected by the treating physician. In any event, the pharmaceutical formulations should provide a quantity of the oligosaccharides of this invention sufficient to effectively treat the patient.

The oligosaccharides may also find use as diagnostic reagents. For example, labeled compounds can be used to locate areas of inflammation or tumor metastasis in a patient suspected of having an inflammation. For this use, the compounds can be labeled with appropriate radioisotopes, for example, ¹²⁵I, ¹⁴C, or tritium.

The oligosaccharide of the invention can be used as an immunogen for the production of monoclonal or polyclonal antibodies specifically reactive with the compounds of the invention. The multitude of techniques available to those skilled in the art for production and manipulation of various immunoglobulin molecules can be used in the present invention. Antibodies may be produced by a variety of means well known to those of skill in the art.

The production of non-human monoclonal antibodies, e.g., murine, lagomorpha, equine, etc., is well known and may be accomplished by, for example, immunizing the animal with a preparation containing the oligosaccharide of the invention. Antibody-producing cells obtained from the immunized animals are immortalized and screened, or screened first for the production of the desired antibody and then immortalized. For a discussion of general procedures of monoclonal antibody production, see, Harlow and Lane, Antibodies, A Laboratory Manual, Cold Spring Harbor Publications, N.Y. (1988).

The following examples are offered solely for the purposes of illustration, and are intended neither to limit nor to define the invention.

### EXAMPLE 1

Expression of CMP-sialic Acid Synthetase/α2,3 Sialytransferase Fusion Protein

This Example describes the use of a single cell type that expresses a CMP-sialic acid synthetase/α2,3-sialyltransferase fusion protein to relatively inexpensively produce 3′-sialyllactose. The approaches are shown schematically in FIG. 1.

A. Cell Overexpresses CMP-sialic acid

A strain of E. coli (EV240) that had been genetically engineered to produce CMP-sialic acid (CMP-NAN) (nanA neuS::Tn10 mutation) was transformed with plasmid DNA that includes a gene that encodes an IPTG-inducible CMP-NAN synthetase/α2,3-sialyltransferase fusion protein. A 1L culture in LB medium was grown to an OD₆₀₀ of 2-3, transferred to 20° C. and induced for 16 hours with IPTG. This culture was harvested and the cell pellet collected by centrifugation. The 7 g cell pellet was then mixed with the following permeabilization solution to initiate the reaction: 250 mM galactose, 250 mM fructose, 10 mM lactose, 100 mM KH₂PO₄, 20 mM MgSO₄.7H₂O (pH7.0) and 1% xylene.

The production of 3′-sialyllactose was monitored by TLC and HPLC. After 43 h, the reaction mixture had produced 2.2 mg/mL of product as determined by TLC (silica, isopropanol:NH₄OH:H₂O (7:1:2) visualized with orcinol); R_{f}=0.8, and by HPLC (BioRad Aminex column HPX-87H, 4 mM sulfuric acid in H₂O); Rₜ=6.3 minutes.

B. Reaction Mixture supplemented with sialic acid and CTP

In this Example, the effect on the reaction in Example 1 of a permeabilization solution that is supplemented with 10 mM NAN and 10 mM CTP is examined. The cell culture described in Example 1 is mixed with a permeabilization solution of 250 mM galactose, 250 mM fructose, 10 mM lactose, 100 mM KH₂PO₄, 20 mM MgSO₄.7H₂O pH7.0, 1% xylene, which is supplemented with 10 mM sialic acid and 10 mM CTP. The reactions are monitored by TLC and HPLC as described in Example 1. Supplementing the reaction mixture with the additional NAN and CTP can result in higher levels of CMP-NAN being available than in the recombinant E. coli cell, and thus lead to higher levels of 3′-sialyllactose production.

C. Use of an E. coli cell that does not overproduce CMP-sialic acid

In this Example the CMP-sialic acid synthetase/α2,3-sialyltransferase fusion protein is expressed in an E. coli strain that does not overproduce CMP-sialic acid.

A 100 mL culture of AD202 E. coli that expressed a fusion protein that includes the catalytic domain of α-2,3-sialyltransferase and CMP sialic acid synthetase was grown at 37° C. on a shaker at 200 rpm. Expression of the fusion protein was induced with IPTG upon the culture's reaching of an OD₆₀₀ equal to 0.85. The culture was incubated at 30° C. overnight. Approximately 2.0g of bacterial cell paste was harvested from this culture.

A solution containing 0.1M HEPES, pH 7.5, was prepared and heated to boiling, after which 1% xylene was added. After the solution cooled to approximately 37° C., 10 mM lactose. 10 mM CTP and 10 mM sialic acid were added. This solution was then thoroughly mixed with the 2.0 g of bacterial cell paste and incubated at 37° C. on a shaker at 150 rpm overnight.

The amount of sialyllactose formed by this reaction was monitored by thin layer chromatography (TLC and HPLC. After 44 h, all of the lactose had been consumed and the concentration of the resultant 3′-sialyllactose was 7.04 mM (70% yield), as determined by TLC (silica; isopropanol/NH₄OH/H₂O (7/1/2), visualized by orcinol, R_{f}=0.8) and HPLC (BioRad Aminex column HPX-87H. 4 mM sulfuric acid in H₂O, Rₜ=6.3 minutes).

### EXAMPLE 2

This Example describes approaches for synthesizing sialylated saccharides in which two organisms are used. Schematic representations of one of these approaches are shown in FIG. 5B. The reaction mixture is similar to that described in Example 1, except that the CTP is produced by an organism such as yeast or Corynebacterium.

A strain of E. coli (EV240) genetically engineered to overexpress CMP-NAN (nanA neuS::Tn10 mutation) is transformed with plasmid DNA encoding an IPTG-inducible CMP-sialic acid synthetase/α2,3-sialyltransferase fusion protein. A culture of these bacteria is grown and induced to make the fusion protein. To initiate the reaction, the cell pellet is added to a solution that contains 1% xylene, 250 mM glucose, 250 mM fructose, 25 mM lactose, 20 mM MgSO₄-7H₂O pH7.0, 100 mM KH₂PO₄ pH7, 10 mM sialic acid, catalytic amounts of CMP. The solution also contains 20% Bakers yeast (w/v). The yeast is used to produce and regenerate the nucleotide CTP used in the sialic acid cycle (fructose, glucose and CMP are used by the yeast to generate the CTP). The CMP-NAN synthetase catalytic domain of the fusion protein that is expressed by the E. coli generates CMP-NAN from the CTP and NAN, and the sialyltransferase catalytic domain then generates 3′sialyllactose.

The reaction is monitored as described in Example 1 and when complete is purified by standard procedures and techniques.

Any organism that can generate CTP can be used in this approach, as can be any organism that overexpresses UTP and also expresses the CMP-synthetase gene (e.g., Corynebacterium). Exogenous myokinase can be added to the reaction mixture, or a yeast that expresses mvokinase can be used to help catalyze the formation of CTP.

### EXAMPLE 3

This Example describes the use of a cell type that contains exogenous genes that encode enzymes that are involved in the synthesis of CMP-sialic acid from GlcNAc. See, FIG. 5B.

A culture of E. coli strain JM101 that expresses the α 2,3-sialyltransferase/CMP sialic acid synthetase fusion protein, GlcNAc 2′-epimerase and sialic acid aldolase is grown and induced to express these enzymes. The cell paste is harvested and, to initiate the reaction, the cell paste is added to a solution that contains GlcNAc, pyruvate, lactose, CTP as well as buffer and other reagents.

The formation of the product of the reaction, 3′-sialyllactose, is monitored by TLC or HPLC and when the reaction is completed, the 3′-sialyllactose is isolated by standard techniques and procedures.

In place of the added CTP, yeast or Corynebacterium (expressing the gene for CTP-synthetase) can be used to produce and regenerate the CTP used in the reaction similar to that described in Example 2.

### EXAMPLE 4

In this Example, an E. coli strain that expresses only the α2,3-sialyltransferase/CMP-sialic acid synthetase fusion protein is used, in conjunction with Bakers yeast, which produces the CTP.

A culture of AD202 bacteria that expresses the α 2,3-sialyltransferase/CMP sialic acid synthetase fusion protein is grown and induced to express this fusion protein. The cell paste is harvested and added to a solution containing 250 MM glucose, 250 mM fructose, 25 mM lactose, 20 mM MgSO₄-7H₂O, pH7.0, 100 mM KH₂PO₄ pH7, 10 mM sialic acid, 1% xylene, 5 mM CMP and 20% (w/v) Bakers yeast to initiate the reaction. The reactions are monitored by TLC and HPLC to follow the production of product. When the reaction is complete the product is purified by standard techniques and procedures.

### EXAMPLE 5

In this Example, the E. coli strain EV5, a strain that overproduces sialic acid, is used. The E. coli strain is transformed with the plasmid that encodes the ST/CMP-SA synthetase fusion protein. Once the culture is grown and the plasmid products expressed, the cells are harvested and the reaction initiated with the addition of a solution containing 250 mM galactose. 250 mM fructose, 10 mM lactose, 100 mM KH₂PO₄, 10 mM CTP, 1% xylene, and 20 mM MgSO₄.7H₂O, pH7.0. The production of 3′-sialyllactose is monitored as described in Example 1 and purified by procedures and protocols known to those skilled in the art.

In place of the added CTP, yeast or Corynebacterium (expressing the gene for CTP-synthetase) can be used to produce and regenerate the CTP in the reaction in a manner similar to that described in Example 2.

### EXAMPLE 6

The reaction in this Example uses a single organism that produces the nucleotide, the nucleotide sugar, and catalyzes the transfer of the sugar to the acceptor saccharide. A culture of Corynebacterium that expresses the α2,3-sialyltransferase/CMP sialic acid synthetase fusion protein and CTP-synthetase is grown and induced to express these enzymes. The cell paste is then be harvested and added to a solution containing lactose, galactose, orotic acid, sialic acid as well as buffer and other reagents. The formation of the product of the reaction, the 3′-sialyllactose, is monitored by TLC or HPLC and when completed, is isolated by standard techniques and procedures.

### EXAMPLE 7

This Example describes the use of an organism that expresses enzymes necessary for production of the trisaccharide Galα1,3Galβ1,4-GlcNAc. The organism includes exogenous genes that encode enzymes of the galactosyltransferase cycle. See, FIG. 5A.

A culture of Corynebacterium that expresses UDP-glucose pyrophosphorylase. UDP-glucose-4′-epimerase, β1,4-galactosyltransferase and the α1,3-galactosyltransferase is grown and induced to express these enzymes. To initiate the reaction, a solution containing GlcNAc, orotic acid, buffer and other reagents is then added to the cell paste. The formation of the product, the trisaccharide Galα1-3Galβ1-4GlcNAc, is monitored by TLC and HPLC and when completed, the product is isolated by standard techniques.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A reaction mixture for producing a product saccharide, wherein the reaction mixture comprises an acceptor saccharide and a first type of plant or microorganism cell that produces: a) a nucleotide sugar, and b) a first recombinant glycosyltransferase that catalyzes the transfer of a sugar from the nucleotide sugar to the acceptor saccharide to form the product saccharide.

2. The reaction mixture of claim 1, wherein the cells are selected from one or more of the group consisting of bacterial cells, yeast cells, fungal cells, and plant cells.

3. The reaction mixture of claim 1, wherein the cells are permeabilized or otherwise disrupted.

4. The reaction mixture of claim 1, wherein the glycosyltransferase is a fucosyltransferase and the nucleotide sugar is GDP-fiucose.

5. The reaction mixture of claim 1, wherein the glycosyltransferase is a sialyltransferase and the nucleotide sugar is CMP-sialic acid

6. The reaction mixture of claim 1, wherein nucleotide sugar is selected from the group consisting of UDP-Gal, UDP-Glc, UDP-Glucuronic acid, UDP-GalNAc, UDP-Galacturonic acid, GDP-mannose.

7. The reaction mixture of claim 1, wherein the first type of cell produces the nucleotide sugar at an elevated level compared to a wild-type cell.

8. The reaction mixture of claim 7, wherein the elevated level of the nucleotide sugar results from a deficiency in the ability of the cell to incorporate the nucleotide sugar into a polysaccharide normally produced by the cell.

9. The reaction mixture of claim 7, wherein the elevated level of the nucleotide sugar is at least 10% higher than the level of the nucleotide sugar produced by the wild-type cell.

10. The reaction mixture of claim 9, wherein the elevated level of the nucleotide sugar is at least 25% higher than the level of the nucleotide sugar produced by the wild-type cell.

11. The reaction mixture of claim 1, wherein the nucleotide sugar is synthesized by an enzymatic pathway that includes one or more enzymes that are expressed from heterologous genes.

12. The reaction mixture of claim 11, wherein the recombinant glycosyltransferase is a sialyltransferase, the nucleotide sugar is CMP-sialic acid and the heterologous gene encodes CMP-sialic acid synthetase.

13. The reaction mixture of claim 12, wherein the acceptor saccharide is lactose and the product saccharide is sialyllactose.

14. The reaction mixture of claim 11, wherein the recombinant glycosyltransferase is a β1,4-GalNAc transferase and the nucleotide sugar is UDP-GalNAc.

15. The reaction mixture of claim 14, wherein the acceptor is lactose and the product saccharide is β1,4-GalNAc-lactose.

16. The reaction mixture of claim 11, wherein the recombinant glycosyltransferase is a galactosyltransferase and the nucleotide sugar is UDP-Gal.

17. The reaction mixture of claim 16, wherein the galactosyltransferase is an α1,3-galactosyltransferase and the product saccharide contains a terminal α1,3-linked galactose residue.

18. The reaction mixture of claim 11, wherein the enzymatic pathway comprises a full or partial sugar nucleotide regeneration cycle.

19. The reaction mixture of claim 18, wherein the nucleotide sugar is UDP-GalNAc and the sugar nucleotide regeneration cycle comprises a set of enzymes selected from the group consisting of:
UDP-GalNAc epimerase, UDP-GlcNAc pyrophosphorylase, GlcNAc-1-kinase, polyphosphate kinase and pyruvate kinase; and
UDP-GalNAc pyrophosphorylase, GlcNAc-1-kinase, polyphosphate kinase and pyruvate kinase.

20. The reaction mixture of claim 19, wherein the reaction mixture further comprises a second cell type that produces a nucleotide that is used as a substrate for the sugar nucleotide regeneration cycle.

21. The reaction mixture of claim 20, wherein the second cell type comprises an exogenous gene that encodes a nucleotide synthetase polypeptide that catalyzes the synthesis of the nucleotide.

22. The reaction mixture of claim 21, wherein the first cell type comprises exogenous genes that encode a) a fusion protein that comprises a polypeptide having 3′-sialyltransferase activity and a polypeptide that has CMP-sialic acid synthetase activity; and b) enzymes that catalyze the synthesis of sialic acid from GlcNAc;
and the second cell type comprises an exogenous gene that encodes CMP-synthetase.

23. The reaction mixture of claim 21, wherein the first cell type is E. coli and the second cell type is yeast or Corynebacterium.

24. The reaction mixture of claim 1, wherein the first type of cell produces a second recombinant glycosyltransferase that catalyzes the transfer of a sugar from the nucleotide sugar to the product saccharide to form a further glycosylated product saccharide.

25. The reaction mixture of claim 24, wherein the nucleotide sugar is UDP-Gal, the first recombinant glycosyltransferase is an β1,4-galactosyltransferase and the second recombinant glycosyttransferase is an α1,3-galactosyltransferase.

26. The reaction mixture of claim 25, wherein the acceptor saccharide is Glc(R)β-O-R¹, wherein R¹ is —(CH₂)ₙ—COX; X is selected from the group consisting of OH, OR², —NHNH₂, R is OH or NAc; R² is a hydrogen, a saccharide, an oligosaccharide or an aglycon group having at least one carbon atom, and n is an integer from 2 to 18.

27. The reaction mixture of claim 25, wherein the UDP-Gal is generated by enzymes that are expressed from exogenous genes that encode UDP-Gal 4′ epimerase and UDP-Glc pyrophosphorylase.

28. The reaction mixture of claim 1, wherein the cell further comprises: a) an enzymatic system for producing at least a second nucleotide sugar, and b) at least a second recombinant glycosyltransferase that catalyzes transfer of a sugar from the second nucleotide sugar to the product sugar.

29. The reaction mixture of claim 28, wherein:
the first recombinant glycosyltransferase is a GlcNAc transferase and the first nucleotide sugar is UDP-GlcNAc; and
the second recombinant glycosyltransferase is a galactosyltransferase and the second nucleotide sugar is UDP-galactose.

30. The reaction mixture of claim 29, wherein the reaction mixture forms lacto-N-neotetraose (LNnT).

31. The reaction mixture of claim 1, wherein the reaction mixture also comprises at least a second type of cell that produces a) a second nucleotide sugar, and b) a second recombinant glycosyltransferase that catalyzes the transfer of the sugar from the second nucleotide sugar to the product saccharide.

32. The reaction mixture of claim 31, wherein the first glycosyltransferase is a galactosyltransferase and the second glycosyltransferase is a GalNAc transferase.

33. The reaction mixture of claim 31, wherein:
the first cell type comprises a recombinant β1,4-GalNAc transferase, a recombinant β1,4-Gal transferase, UDP-GalNAc and UDP-Gal; and
the second cell type comprises a recombinant α2,3-sialyltransferase and CMP-sialic acid.

34. The reaction mixture of claim 33, wherein the CMP-sialic acid is produced from CTP and GlcNAc by an enzymatic system in the second cell type that includes recombinant enzymes CMP-sialic acid synthetase, GlcNAc epimerase, NeuAc aldolase, and CMP-synthetase.

35. The reaction mixture of claim 33, wherein the acceptor saccharide is lactosylceramide or lyso-lactosylceramide and the product saccharide is ganglioside GM₂.

36. The reaction mixture of claim 33, wherein the second cell type further comprises a recombinant α2,8-sialyltransferase.

37. The reaction mixture of claim 36, wherein the acceptor is lactosylceramide or lyso-lactosylceramide and the product saccharide is GD₂.

38. The reaction mixture of claim 1, wherein the reaction mixture also comprises a second type of cell that produces a nucleotide from which is synthesized the nucleotide sugar produced by the first type of cell.

39. The reaction mixture of claim 38, wherein nucleotide produced by the second cell type and the corresponding nucleotide sugar are selected from the group consisting of:
UTP: UDP-Gal, UDP-GalNAc, UDP-GlcNAc, UDP-Glc, UDP-glucuronic acid, or UDP-galacturonic acid;
GTP: GDP-Fuc; and
CTP: CMP-sialic acid.

40. A cell that produces a product saccharide, wherein the cell comprises:
a) a recombinant gene that encodes a glycosyltransferase;
b) an enzymatic system for forming a nucleotide sugar that is a substrate for the glycosyltransferase; and
c) an exogenous saccharide acceptor moiety;
wherein the glycosyltransferase catalyzes the transfer of a sugar from the nucleotide sugar to the acceptor moiety to produce the product saccharide.

41. The cell of claim 40, wherein the enzymatic system for forming a nucleotide sugar comprises cycle enzymes for regenerating the nucleotide sugar.

42. The cell of claim 40, wherein the recombinant gene that encodes a glycosyltransferase is a heterologous gene.

43. The cell of claim 40, wherein the cell forms the nucleotide sugar at an elevated level compared to a wild-type cell.

44. The cell of claim 43, wherein the elevated level of nucleotide sugar results from a deficiency in the ability of the cell to incorporate the nucleotide sugar into a polysaccharide normally produced by the cell.

45. The cell of claim 44, wherein the deficiency is due to a reduced level of a polysaccharide glycosyltransferase activity.

46. The cell of claim 40, wherein the product saccharide is produced at a concentration of at least about 1 mM.

47. The cell of claim 40, wherein the enzymatic system for forming a nucleotide sugar comprises an enzyme encoded by a heterologous gene.

48. The cell of claim 47, wherein the enzyme encoded by the heterologous gene is one or more of:
a GDP-mannose dehydratase, a GDP-mannose 3,5-epimerase, and a GDP-mannose 4-reductase;
a UDP-galactose 4′ epimerase;
a UDP-GalNAc 4′ epimerase;
a CMP-sialic acid synthetase;
a pyrophosphorylase selected from the group consisting of a UDP-Glc pyrophosphorylase, a UDP-Gal pyrophosphorylase, a UDP-GalNAc pyrophosphorylase, a GDP-mannose pyrophosphorylase, and a UDP-GlcNAc pyrophosphorylase;
a kinase selected from the group consisting of myokinase, pyruvate kinase, acetyl kinase, creatine kinase; and
pyruvate decarboxylase.

49. The cell of claim 48, wherein the nucleotide sugar is GDP-fucose.

50. A cell that produces a sulfated polysaccharide, the cell comprising:
a heterologous gene that encodes a sulfotransferase; and
an enzymatic system that produces PAPS.

51. The cell of claim 50, wherein the sulfated polysaccharide is selected from the group consisting of heparin sulfate and carragenin.

52. The cell of claim 50, wherein the enzymatic system that produces PAPS comprises one or more enzymes that are expressed from exogenous genes.

53. A method of producing a product saccharide, the method comprising contacting a microorganism or plant cell with an acceptor saccharide, wherein the cell comprises:
a) an enzymatic system for forming a nucleotide sugar; and
b) a recombinant glycosyltransferase which catalyzes the transfer of a sugar from the nucleotide sugar to the acceptor saccharide to produce the product saccharide.

54. The method of claim 53, wherein the glycosyltransferase is encoded by a heterologous gene.

55. The method of claim 53, wherein the glycosyltransferase is encoded by a gene that is endogenous to the cell and is produced by the cell at an elevated level compared to a wild-type cell.

56. The method of claim 53, wherein the product saccharide is produced at a concentration of at least about 1 mM.

57. The method of claim 53, wherein the cell is permeabilized.

58. The method of claim 53, wherein the cell is an intact cell.

59. The method of claim 53, wherein the enzymatic system for forming a nucleotide sugar comprises an enzyme that is encoded by a heterologous gene.

60. The method of claim 59, wherein the enzyme encoded by the heterologous gene is one or more of:
a GDP-mannose dehydratase, a GDP-4-keto-6-deoxy-D-mannose 3,5-epimerase, and a GDP-4-keto-6-deoxy-L-glucose 4-reductase;
a UDP-galactose 4′ epimerase;
a UDP-GalNAc 4′ epimerase;
a CMP-sialic acid synthetase;
a pyrophosphorylase selected from the group consisting of a UDP-Glc pyrophosphorylase, a UDP-Gal pyrophosphorylase, a UDP-GalNAc pyrophosphorylase, a GDP-mannose pyrophosplhorylase, and a UDP-GlcNAc pyrophosphorylase; a kinase selected from the group consisting of myokinase, pyruvate kinase, acetyl kinase, creatine kinase; and
pyruvate decarboxylase.

61. The method of claim 59, wherein the enzyme for forming a nucleotide sugar and the glycosyltransferase are expressed as a fusion protein.

62. The method of claim 61, wherein the fusion protein comprises a CMP-sialic acid synthetase activity and a sialyltransferase activity.

63. The method of claim 61, wherein the fusion protein comprises a galactosyltransferase activity and a UDP-Gal 4′ epimerase activity.

64. The method of claim 61, wherein the fusion protein comprises a GalNAc transferase activity and a UDP-GlcNAc 4′ epimerase activity.

65. The method of claim 53, wherein the nucleotide sugar is GDP-fucose and the glycosyltransferase is a fucosyltransferase.

66. The method of claim 53, wherein the cell forms the nucleotide sugar at an elevated level compared to a wild-type cell.

67. The method of claim 66, wherein the elevated level of nucleotide sugar results from a deficiency in the ability of the cell to incorporate the nucleotide sugar into a polysaccharide normally produced by the cell.

68. The method of claim 67, wherein the deficiency is due to a reduced level of a polysaccharide glycosyltransferase activity.

69. The method of claim 53, wherein the cell/nucleotide sugar are selected from the group consisting of:
Azotobacter vinelandii/GDP-Man;
Pseudomonas sp./UDP-Glc and GDP-Man;
Rhizobium sp./UDP-Glc, UDP-Gal, GDP-Man;
Erwinia sp./UDP-Gal, UDP-Glc;
Escherichia sp./UDP-GlcNAc, UDP-Gal, CMP-NeuAc, GDP-Fuc;
Klebsiella sp./UDP-Gal, UDP-GlcNAc, UDP-Glc, UDP-GlcNAc;
Hansenula jadinii/ GDP-Man, GDP-Fuc;
Candida famata/UDP-Glc, UDP-Gal, UDP-GlcNAc;
Saccharomyces cerevisiae/UDP-Glc, UDP-Gal, GDP-Man, GDP-GlcNAc; and
X. campesti/UDP-Glc, GDP-Man.

70. The method of claim 53, wherein the cell is Azotobacter vinelandii, the nucleotide sugar is GDP-mannose, the acceptor saccharide is lactose, the glycosyltransferase is manmosyl transferase, and the product saccharide is mannosyl lactose.

71. The method of claim 53, wherein the cell is E. coli, the nucleotide sugar is CMP-sialic acid, the acceptor saccharide is lactose, the glycosyltransferase is a sialyltransferase, and the product saccharide is sialyllactose.
